SEQUENCE LISTING (2 sequences)

SEQ ID NO: 1 - DNA, 20 bp, Homo sapiens
cctccgcggccatgaacttt

SEQ ID NO: 2 - DNA, 22 bp, Homo sapiens
tctttccggatccgagatctgg

(19) United States
(12) Patent Application Publication
(10) Pub. No.: US 2009/0155266 A1
(43) Pub. Date: Jun. 18, 2009

(54) Methods and Compositions Relating to Vascular Endothelial Growth Factor and TH2 Mediated Inflammatory Diseases

(75) Inventor: Jack A. Elias, Woodbridge, CT (US)

Correspondence Address: COOLEY GODWARD KRONISH LLP;ATTN: Patent Group, Suite 1100, 777 - 6th Street, NW, WASHINGTON, DC, 20001, US

(73) Assignee: YALE UNIVERSITY, New Haven, CT (US)

(21) Appl. No.: 10/586,254

(22) PCT Filed: Jan. 18, 2005
(86) PCT No.: PCT/US05/01500

§ 371 Date: Oct. 31, 2008

Related U.S. Application Data
(60) Provisional application No. 60/537,195, filed on Jan. 16, 2004.
(60) Provisional application No. 60/574,086, filed on May 25, 2004.

(51) Int. Cl. A61K 39/395; A61K 31/7052; A61K 38/02; A61P 29/00; A61K 38/43; G01N 33/68

(52) U.S. Cl. 424/134.1; 424/158.1; 514/44; 514/2; 424/94.1; 436/86; 435/29

(57) ABSTRACT

The present invention includes compositions and methods for the treatment of a Th2 mediated inflammatory disease, relating to inhibiting a VEGF. The invention further includes methods to identify new compounds for the treatment of a Th2 mediated inflammatory disease, including, but not limited to, asthma and the like. This is because the present invention demonstrates, for the first time, that expression of VEGF induces asthma-like phenotype and that inhibiting VEGF reverses the phenotype. Thus, the invention relates to the novel discovery that inhibiting VEGF treats and prevents an Th2 mediated inflammatory disease.

### RELATED APPLICATIONS

The present application is a U.S. National Phase Application of International Application PCT/US2005/001500 (filed Jan. 18, 2005) which claims the benefit of U.S. Provisional Application No. 60/537,195 (filed Jan. 16, 2004) and U.S. Provisional Application No. 60/574,086 (filed May 25, 2004), all of which are herein incorporated by reference in their entirety for all purposes.

### STATEMENT REGARDING FEDERALLY SPONSORED RESEARCH OR DEVELOPMENT

This invention was supported in part by funds obtained from the U.S. Government (National Institutes of Health Grant Numbers HL-64642, HL-61904, HL-56389, HL-24136, and HL-59157) and the U.S. Government may therefore have certain rights in the invention.

### BACKGROUND OF THE INVENTION

The lung is unique amongst mucosal compartments in that it is constantly exposed to airborne particulates. In normal individuals, the immune response is able to differentiate harmless agents that should not induce sensitization and potentially injurious pathogens against which an immune response is warranted. In contrast, the lungs of atopic asthmatics manifest an enhanced ability to sensitize and mount pathologic T-helper cell type 2 (Th2) responses after exposure to largely innocuous allergens (Schwarze et al. 2002; Eisenbarth et al., 2002; Umetsu et al., 2002). Infection elicited innate immune responses are known to play an essential role in the development of adaptive Th2 immunity (Schwarze et al. 2002; Eisenbarth et al., 2002). This is nicely illustrated by respiratory syncytial virus (RSV) which enhances aeroallergen sensitization and contributes to asthma disease acquisition (Schwarze et al. 2002; Sigurs et al., 2000). It has been proposed that antiviral innate responses contribute to the generation and/or maintenance of adaptive Th2 immunity by increasing mucosal permeability and altering local dendritic cells (DC) Schwarze et al. 2002). The validity of these assumptions has not been tested and the mediators of these effects have not been defined.

On the other hand, mediators of Th2 inflammation not resulting from innate immune responses but from airborne antigens have been well documented. The immune cells and mediators implicated in asthmatic inflammation, for example, include IgE, mast cells, basophils, eosinophils, T cells, interleukin-4 (IL-4), IL-5, IL-9, IL-13 and other cytokines (Bradding et al., 1994, Am. J. Respir. Cell Mol. Biol. 10:471-480; Bradding et al., 1997, Airway Wall Remodeling in Asthma, CRC Press, Boca Raton, Fla.; Nicolaides et al., 1997, Proc. Natl. Acad. Sci. USA 94:13175-13180; Wills-Karp, 1998, Science 282:2258-2260; Hamid et al., 1991, J. Clin. Invest. 87:1541-1546; Kotsimbos et al., 1996, Proc. Assoc. Am. Physicians 108:368-373). Of these immune cells and mediators, the role of Th2 cells and Th2 cytokines such as GM-CSF, IL-3, IL-4, IL-5 and IL-13 is proving to be increasingly important, as they are believed to be responsible for initiation and maintenance of airway inflammation, as well as vital to B cell regulation, eosinophil function, mucus responses, and stimulation of airway remodeling (Elias et al., 1999, J. Clin. Invest. 104:1001-1006; Ray et al., 1999, J. Clin. Invest. 104:985-993).

Two prominent cytokines, IL-4 and IL-13, in particular, are believed to play an important role in the inflammation and airway remodeling of asthma and other pulmonary diseases. IL-4 and IL-13 are similar in that they are both produced by the same subset of Th2 helper T cells, have overlapping effector profiles, and share a receptor component and signaling pathways. The role of IL-13 over IL-4 in airways hyperresponsiveness (AHR), eosinophil recruitment, mucus overproduction, and other symptoms of asthma has been documented (Wills-Karp, 1998, Science 282:2258-2260, Grunig et al. 1998, Science 282:2261-2263).

Overexpression of IL-13 in the murine lung results in eosinophil, lymphocyte, and macrophage rich inflammation, mucus metaplasia, airway fibrosis, and AHR after methacholine challenge (Zheng et al., 1999 J. Clin. Invest. 103:779-788). Further, polymorphisms in both the IL-13 promoter and the coding region have been associated with the asthmatic phenotype (Heinzmann et al., 2000, Hum. Mol. Genet. 9:549-559).

Exaggerated Th2 inflammation and airway remodeling are characteristic of and cornerstones in the pathogenesis of asthma (Wills-Karp et al., 2003; Elias et al., 1999; 2003). In keeping with the importance of neovascularization in inflammation and remodeling, a number of investigators have characterized the vascular response in asthmatic tissues. These studies showed prominent increases in vessel number, vessel size, vascular surface area and vascular leakage and correlations between these alterations and disease severity (Vrugt et al., 2000; Slavato et al., 2001; Li et al., 1997: Lee et al., 2001; Orsida et al., 2001; Hogg et al., 1999; Hoshino et al., 2001; Charan et al., 1997). As a result, it has been assumed that asthmatic inflammation stimulates the growth of new blood vessels (Hogg et al., 1999; Charan et al., 1997) and that these vascular alterations contribute to the airways obstruction and/or airways hyperresponsiveness (AHR) in this disorder (Charan et al., 1997; Thurston et al., 2000; Antony et al., 2002).

Vascular endothelial growth factor (VEGF) was originally described as vascular permeability factor (VPF) based on its ability to generate tissue edema (Senger et al., 1993). It has subsequently been appreciated to be a multifunctional angiogenic regulator that stimulates epithelial cell proliferation, blood vessel formation and endothelial cell survival (Gerber et al., 1998; Clauss et al., 2000). VEGF is a homodimeric glycoprotein consisting of two 23 kD subunits. Four different monomeric isoforms of VEGF exist resulting from alternative splicing of mRNA. These include two membrane bound forms (VEGF₂₀₆ and VEGF₁₈₉) and two soluble forms (VEGF₁₆₅ and VEGF₁₂₁). Three receptors for VEGF have been described: VEGFR-1 (Flt-1) binds VEGF; VEGFR-2 (Flk-1/KDR) binds VEGF; and VEGFR-3 (Flt-4) appears to be specific for VEGF-C (Neufeld et al., FASEB J., 13:9-22, 1999). In addition, other VEGF receptors such as neuropillins have been identified.

Exaggerated levels of VEGF have been detected in tissues and biologic samples from patients with asthma where they correlate directly with disease activity (Lee et al., 2001) and inversely with airway caliber and airway responsiveness (Hoshino et al., 2001; Kanazawa et al., 2002; Asai et al., 2002). VEGF has been postulated to contribute to asthmatic tissue edema via its VPF effect (Charan et al., 1997; Thurston et al., 2000: Antony et al., 2002). Surprisingly, the role of VEGF in the pathogenesis of other aspects of the asthmatic phenotype and the effector profile of VEGF in the lung have not been defined. Even the contribution of VEGF to asthmatic vascular alterations is not clear because VEGF has been reported to lack angiogenic properties in the respiratory tract (Kaner et al., 2000; Partovian et al., 2000).

Since VEGF plays a critical role in vascular formation at the developmental stage and the pathological neovascularization associated not only with asthma but also with diabetes, rheumatoid arthritis, retinopathy, and the growth of solid tumors, angiogenesis, VEGF is presently a target of efforts to develop useful, novel drugs.

Humanized neutralizing antibodies have been shown to interact with VEGF near the KDR and Flt-1 binding sites (Kim, K. J., et al., (1993) Nature 362, 841-844; Muller, Y. A., et al. (1997) Proc. Nat. Acal. Sci. 94, 7192-7197; Muller, Y. A. et al., (1998) Structure 6:1153-1167; U.S. Pat. No. 5,855,866), and SELEX-derived RNA molecules (Jellinek, D et al., (1994) Biochemistry 33:10450-10456; U.S. Pat. No. 5,859,228), that target VEGF, suppress tumor growth that is dependent on vascularization of adjacent normal tissue (Plate, K. H. et al., (1994) Brain-Pathol., 4:207-218). Anti KDR monoclonal antibodies inhibited VEGF induced signaling and demonstrated high anti-tumor activity (Witte et al., (1998) Cancer & Metast. Reviews 17:155-161; U.S. Pat. No. 5,840,301). Soluble Fit receptor (U.S. Pat. No. 5,861,484), fragments of VEGF (U.S. Pat. No. 5,240,848) have been shown to inhibit factor/receptor interaction and angiogenesis in vivo.

Anti VEGF antisense oligonucleotides were designed to inhibit VEGF expression and VEGF induced neovascularization (U.S. Pat. No. 5,641,756).

VEGF antagonists also include small molecules such as chemical compounds. For example, Hennequin et al. in J. Med. Chem. 42, 5369-5389 (1999) disclose certain quinazolines, quinolines and cinnolines as being useful as antagonists of VEGF receptors. See also Annie et al., Journal of Acquired Immune Deficiency Syndromes and Human Retrovirology 17, A41 (1998). Additionally, App et al. (U.S. Pat. No. 5,849,742) disclose small molecule derivatives of quinazoline, quinoxiline, substituted aniline, isoxazoles, acrylonitrile and phenylacrylonitrile compounds which act as tyrosine kinase antagonists.

### BRIEF SUMMARY OF THE INVENTION

The present invention is based on the discovery discussed herein that VEGF is a critical mediator in asthma and Th2 inflammation. VEGF₁₆₅ was overexpressed in the airway of transgenic mice and the role of VEGF in antigen-induced Th2 inflammation was evaluated. The studies provided herein demonstrate that VEGF is a potent stimulator of bronchial angiogenesis and edema, inflammation, vascular remodeling, parenchymal remodeling and physiologic dysregulation. The present invention establishes a link between innate and adaptive Th2 immunity by demonstrating that VEGF enhances antigen sensitization and Th2 inflammation and demonstrates that epithelial and Th2 cell-derived VEGF play central roles in Th2 inflammation and cytokine elaboration.

The present invention provides methods of treating a Th2 mediated inflammatory disease in a mammal wherein the disease is associated with an increased level of VEGF. The methods comprise administering an effective amount of VEGF antagonist to the mammal, thereby treating the inflammatory disease in the mammal. The VEGF antagonist can be selected from the group consisting of a chemical compound, a small molecule inhibitor, an antibody, a ribozyme, a nucleic acid (including but not limited to interfering double stranded RNA, also known as dsRNAi and siRNA), an antisense nucleic acid molecule, and soluble receptors that may or may not be attached to antibody fragments.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIG. 1A through 1F, depicts the constructs used to generate dual transgene (+) (TG) mice and the vascular consequences of transgenic VEGF expression. FIG. 1A shows the constructs used to make CC10-rtTA-VEGF transgenic mice. FIG. 1B illustrates the vascular effects of VEGF in the central trachea and left mainstem bronchus using Lycopersicon esculentum lectin staining. Upper left=part of wholemount of cervical trachea from WT mouse on dox water; upper right=left mainstem bronchus from wild type (WT) mouse on normal water; middle left and lower left=left mainstem bronchi from TG mice on dox for 3 and 5 days respectively; middle right and lower right=enlarged areas from middle and lower right panels respectively. Arrows in middle right panel show endothelial sprouts. Scale bar in lower right panel applies to all panels and represents 100 μm in upper right, upper left and middle left panels, and 50 μm in middle right and lower right panels. FIG. 1C shows toluidine blue-stained sections of bronchi. Blood vessels (arrows) and subepithelial elastic lamina (arrowheads). In WT mouse on normal water (top) dark Clara cells are separated by lighter ciliated cells. The middle and lower panels are from mainstem and terminal bronchus respectively from 7 day dox-treated TG mouse. They show an increased number of subepithelial blood vessels compared to WT. Smooth muscle bundles also appear enlarged compared to WT. Arrowhead in bottom panel indicates intraepithelial blood vessel. Scale bar in bottom panel applies to all panels and represents 20 μm. FIG. 1D are electron micrographs showing that compared to WT mouse on normal water (top left), in TG mouse on dox water for 7 days (upper right), numerous blood vessels (arrows) are at the base of the epithelium. Arrowhead in upper right panel indicates vacuole in epithelial cell. Endothelial cell in lower left panel has thick wall, while that in lower right panel has thin wall and pericyte processes (arrowheads). Scale bar in lower right panel applies to all panels and represents 25 g/m in upper and 10 μm in bottom panels. FIGS. 1E and 1F show respectively the results of using Evans Blue dye extravasation (EBD) and wet/dry ratios to compare the permeability in WT and TG mice on normal or dox water for 7 days (*P<0.01 versus the other 3 groups). All histologic and quantitative evaluations are from a minimum of 6 animals. Values represent the mean±SEM.

FIG. 2, comprising FIG. 2A through FIG. 2G, depicts structural alterations in TG mice. WT and TG mice were randomized to normal water or dox water at 1 month of age and evaluated at intervals thereafter. FIG. 2A shows the inflammatory and morphologic changes of WT mice and TG mice by H&E stains (left) and BAL analysis (right). In the H&E stains upper left and upper middle are from WT mice on normal water and dox water respectively for 2 months; upper right is from TG mice on normal water for 2 months and lower left, lower middle and lower right are from TG mice on dox for 7 days, 1 month, and 2 months respectively (all 10× original magnification). In the BAL analysis WT and TG mice on dox for one month are compared (*P<0.05 versus same cell population in WT littermate controls). In FIG. 2B the levels of mucus (top) and mucin and gob-5 gene expression (bottom) were evaluated with D-PAS staining and RT-PCR evaluations respectively (The arrows highlight (+) staining epithelial cells, 20×). FIG. 2B shows the levels of mucus (top) and mucin and gob-5 gene expression (bottom) with D-PAS staining and RT-PCR evaluations respectively. The arrows highlight (+) staining epithelial cells (20×). FIG. 2C is a comparison of α-smooth muscle actin positive cells and tissue fibrosis from WT and TG mice. In the former, immunohistochemistry (IHC) with antibodies against α-smooth muscle actin was employed to compare lungs on dox for one month (top panels). To assess collagen, Mallory's trichrome staining was undertaken comparing WT and TG mice on dox for 4 months (bottom panels). The enlarged muscle bundles and airway fibrosis in lungs from TG mice on dox are readily appreciated. Abnormalities in α-smooth muscle actin or trichrome staining in WT mice on normal or dox water and TG mice on normal water were not appreciated. FIG. 2D shows the Sircol collagen assays which were used to compare the collagen content of WT and TG mice on dox for 4 months. Abnormalities in collagen content in lungs from WT mice on normal or dox water and TG mice on normal water were not appreciated. FIG. 2E shows TGF-β₁ ELISA evaluations of acid-activated (left) and untreated (right) BAL fluids which are used to compare the total and spontaneously active TGF-β₁ respectively in lungs from WT mice and TG mice on dox for 4 months (*P<0.01). FIG. 2F shows the methacholine responsiveness of WT and TG mice on dox for 7 days is compared (*P<0.01). In each Figure histologic evaluations are representative of n≧6 and quantitated values represent the mean±SEM of evaluations in a minimum of 5 animals.

FIG. 3, comprising FIG. 3A through FIG. 3D, shows the role of IL-13 in the VEGF phenotype. FIG. 3A shows the levels of IL-13 mRNA, as compared to the levels of mRNA encoding β-actin, in whole lung RNA from WT and TG mice on dox for 1 month. FIG. 3B through 3D shows the ability of VEGF to induce mucus metaplasia, pulmonary fibrosis and AHR respectively in mice with wild type (+/+) and null mutant (−/−) IL-13 loci. The arrows in 3B highlight the + staining cells in IL-13 (+/+) mice, in FIG. 3C, *P<0.01 versus transgene (−) animals, in FIG. 3D *P<0.05 versus transgene (−) controls. In each panel histologic evaluations are representative of n≧5 and quantitated values represent the mean±SEM of evaluations in a minimum of 5 animals.

FIG. 4, comprising FIG. 4A through FIG. 4F, shows the effects of VEGF on antigen sensitization, Th2 inflammation and DC. In FIGS. 4A and 4B, WT and TG mice on dox for 2 weeks were challenged with ovalbumin (OVA) or vehicle control (OVA(−)) via an intranasal route as described in the Methods. OVA-induced spleen cell proliferation (2A) and OVA-specific IgG₁ (2B) were then assessed (*P<0.01). In FIGS. 4C and 4D, WT and TG mice on dox water for 2 weeks were challenged with OVA as noted above. One week later they were rechallenged with OVA and BAL cellularity (4C) and AHR (4D) were evaluated 48 hours later (In FIG. 4C, *P<0.001 versus eosinophil recovery in all other groups; In FIG. 4D *P<0.01 versus WT controls and **P<0.05 versus TG mouse challenged with vehicle (VEGF TG/OVA(−)). In FIG. 4E, lung cells were isolated from WT mice and TG mice on dox for 7 days and staining with CD11c and MHC II was evaluated by FACS. In FIG. 4F lung cells were isolated from WT and TG mice on dox for 7 days and staining with the noted antibodies (grey) and isotype control antibodies (transparent histograms) was undertaken. In each Figure quantitated values represent the mean±SEM of evaluations in a minimum of 5 animals and FACS evaluations are representative of n≧3.

FIG. 5, comprising FIG. 5A through 5H shows the role of VEGF in Th2 mediated inflammation. WT mice were sensitized and boosted with OVA and alum. They were then challenged with OVA in the presence and absence of the VEGF inhibitor SU1498 (SU) as described in the Methods. In FIG. 5A, bronchioalveolar lavage (BAL) cell recovery is quantitated. Each value represents the mean±SEM of a minimum of 6 animals (*P<0.01 versus OVA challenged mice in the absence of inhibitor). In FIGS. 5B and 5C, the histology (H&E stain) (20×) and methacholine responsiveness respectively of sensitized mice that received vehicle (PBS) and OVA in the presence and absence of SU1498 are compared. In FIG. 5D, IHC is used to localize the VEGF in the lungs from mice challenged with OVA (left). On the left, the staining with anti-VEGF and an isotype control antibody in OVA sensitized and challenged mice and controls was compared. On the right, the staining with anti-VEGF (red) and anti-CD3 (blue) in sensitized and challenged mice was compared. In FIG. 5E, naïve CD4 cells and polarized Th1 and Th2 cells were generated in vitro and, after washing, were cultured in the presence of antigen (OVA₃₂₃₋₃₂₉), and antigen presenting cells (APC). The levels of VEGF in supernatants from cultures containing antigen, APC, naïve CD4 cells and or polarized Th1 or Th2 cells were evaluated by ELISA. Each value represents the mean±SEM of a minimum of 3 cell preparations (*P<0.05). FIG. 5F compares the levels of Th2 cytokines in BAL from mice sensitized with OVA and alum and challenged with OVA in the presence and absence of SU1498. Each value represents the mean±SEM of a minimum of 6 animals (P<0.01 versus OVA sensitized and challenged mice in the absence of SU1498). In FIG. 5G the mRNA encoding VEGF in lungs from wild type (WT) and T-bet(−/−) mice was compared. In FIG. 5H, the BAL cell recovery in WT mice and T-bet (−/−) mice treated with SU1498 or vehicle control was compared. These noted values represent the mean±SEM of evaluations in 6 animals (*P<0.01).

FIG. 6, comprising FIG. 6A through 6E, depicts the reversibility of VEGF effects. WT and TG (+) mice were placed on dox water for 2 weeks. They were then sacrificed or placed back on normal water. The later mice were evaluated 2 weeks (FIGS. 6A-6C) or 4 weeks (FIGS. 6D and 6E) later. The effects of doxycycline removal and transgene deactivation or BAL cell recovery (6A) mucus metaplasia (6B), vascular remodeling (6C), smooth muscle hyperplasia (6D), and AHR (6E) are illustrated. The values in panel a represent the mean±SEM of experiments with a minimum of 5 mice (p<01 vs. TG (+) on dox for 2 weeks). In FIG. 6C evaluations were undertaken with WT mice, (top), TG (+) mice on dox for 2 weeks (middle) and TG (+) mice on dox for 2 weeks followed by normal water for 2 weeks (bottom). L-esculentum evaluations are on the (left) and immunohistochemistry for CD 31 is on the right. The arrows point to CD31 (+) endothelial cells. In FIG. 6E, the noted values are the mean±SEM of evaluations in a minimum of 6 animals.

FIG. 7 depicts Western blot analysis of anti-human VEGF antibodies in serum from WT and TG(+) mice. Bovine serum ovalbumin (100 ng) and recombinant VEGF (100 ng) containing BSA were evaluated via electrophoresis and Western blotting using serum from TG (−), TG(+) (pooled sera 3 mice each) and anti-VEGF antibody. As shown, sera of TG(−) and TG(+) mice did not react with recombinant VEGF.

FIG. 8 shows VEGF expression of Th1 and Th2 cells in the absence of antigen presenting cells (APC). Th1 and Th2 cells were generated as described in the text. The cells were then restimulated with phorbol (PMA) and ionomycin and supernatant VEGF levels were measured by ELISA. The noted experiment is representative of N=3.

FIG. 9, comprising FIG. 9A through 9C, shows the role of VEGF in Th2 inflammation. WT mice were sensitized and boosted with OVA and alum. They were then challenged with OVA in the presence and absence of VEGF_{R1R2} Trap (Vtrap) or control antibody. In FIG. 9A, BAL cell recovery was quantitated. Each value represents the mean±SEM of a minimum of 8 animals (p<0.01 vs. OVA challenged mice in the absence of inhibitor). In FIGS. 9B and 9C, methacholine responsiveness (FIG. 9B) and tissue histology (H&E stain); 10× (FIG. 9C) of sensitized mice that received vehicle or OVA in the presence or absence of Vtrap or its control antibody (Fc).

FIG. 10, comprising FIG. 10A through 10C, shows localization of VEGF expression in epithelial cells of OVA sensitized and challenged mice. Fluorescent immunohistochemistry was used to identify the location of C10 (red, FIGS. 10A and 10C) and VEGF (green, FIGS. 10B and 10C). Double immunohistochemistry is illustrated in FIG. 10C. VEGF was expressed in most of the CC10 (+) cells (arrows) and also in cells that did not express C10 (green only) (40×).

### DETAILED DESCRIPTION OF THE INVENTION

All publications and patent applications herein are incorporated by reference to the same extent as if each individual publication or patent application was specifically and individually indicated to be incorporated by reference.

The following description includes information that may be useful in understanding the present invention. It is not an admission that any of the information provided herein is prior art or relevant to the presently claimed inventions, or that any publication specifically or implicitly referenced is prior art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

The invention provides methods of treating a Th2 mediated inflammatory disease in a mammal where the disease is associated with, or mediated by, expression of VEGF. The methods comprise administering a VEGF antagonist to the mammal. As the data disclosed elsewhere herein demonstrate, increased level of VEGF is associated with, and/or mediates a Th2 mediated inflammatory disease including, but not limited to, asthma, chronic obstructive pulmonary disease, interstitial lung disease, chronic obstructive lung disease, chronic bronchitis, eosinophilic bronchitis, eosinophilic pneumonia, pneumonia, inflammatory bowel disease, atopic dermatitis, atopy, allergy, allergic rhinitis, idiopathic pulmonary fibrosis, scleroderma, emphysema, and the like.

The data disclosed herein demonstrate that increased expression, presence and/or activity of VEGF is associated with and/or mediates various inflammatory disease-associated etiologies including, but not limited to, inflammation, parenchymal and vascular remodeling, edema, mucus metaplasia, myocyte hyperplasia, tissue fibrosis, and airways hyperresponsiveness, and the like.

The data disclosed herein demonstrate, surprisingly, that administering a VEGF antagonist, such as, but not limited to, SU1498, provides a therapeutic benefit and treats the disease. Further, the data disclosed herein demonstrate, for the first time, that administration of an antagonist of VEGF, e.g., a VEGF small molecule antagonist, provides a therapeutic effect and treats the disease. Indeed, the data demonstrate that administration of a VEGF antagonist before onset of the disease state serves to prevent the disease. Accordingly, the present invention provides a novel method whereby administration of a VEGF antagonist in a mammal afflicted with a Th2 mediated inflammatory disease treats and/or prevents the disease when the disease is mediated by, or associated with, VEGF. The invention further provides a method of treating a disease or disorder/condition associated with VEGF-induced neovascularization or angiogenesis in the lung.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

By the term “applicator” as the term is used herein, is meant any device including, but not limited to, a hypodermic syringe, a pipette, an intravenous infusion, topical cream and the like, for administering the VEGF antagonist chemical compound, an antibody, nucleic acid, protein, and/or composition of the invention to a mammal.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting there from. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “fragment” as applied to a nucleic acid, may ordinarily be at least about 20 nucleotides in length, typically, at least about 50 nucleotides, more typically, from about 50 to about 200 nucleotides, preferably, at least about 200 to about 300 nucleotides, even more preferably, at least about 300 nucleotides to about 400 nucleotides, yet even more preferably, at least about 400 to about 500, even more preferably, at least about 500 nucleotides to about 600 nucleotides, yet even more preferably, at least about 600 to about 700, even more preferably, at least about 700 nucleotides to about 800 nucleotides, yet even more preferably, at least about 800 to about 900, even more preferably, at least about 900 nucleotides to about 1000 nucleotides, yet even more preferably, at least about 1000 to about 1100, even more preferably, at least about 1100 nucleotides to about 1200 nucleotides, yet even more preferably, at least about 1200 to about 1300, even more preferably, at least about 1300 nucleotides to about 1400 nucleotides, yet even more preferably, at least about 1400 to about 1500, at least about 1500 to about 1550, even more preferably, at least about 1550 nucleotides to about 1600 nucleotides, yet even more preferably, at least about 1600 to about 1620 and most preferably, the nucleic acid fragment will be greater than about 1625 nucleotides in length.

As used herein, the terms “gene” and “recombinant gene” refer to nucleic acid molecules comprising an open reading frame encoding a polypeptide of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

A “Th2 mediated inflammatory disease” is used herein to refer to a state in which there is a response to tissue damage, cell injury, an antigen, and/or an infectious disease. In some cases, causation will not be able to be established. The symptoms of inflammation may include, but are not limited to cell infiltration and tissue swelling. Disease states contemplated under the definition of inflammatory disease include asthma, chronic obstructive pulmonary disease, interstitial lung disease, chronic obstructive lung disease, chronic bronchitis, eosinophilic bronchitis, eosinophilic pneumonia, pneumonia, inflammatory bowel disease, atopic dermatitis, atopy, allergy, allergic rhinitis, atopic dermatitis, idiopathic pulmonary fibrosis, scleroderma, parasitic infection and its consequences, emphysema, and the like. Some of these diseases are associated with and/or mediated by the exaggerated production of IL-13, IL-4, IL-5 and or IL-9. The diseases also include those that future research will show are mediated by such a pathologic response.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids, which have been substantially purified from other components, which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence.

Preferably, when the nucleic acid encoding the desired protein further comprises a promoter/regulatory sequence, the promoter/regulatory sequence is positioned at the 5′ end of the desired protein coding sequence such that it drives expression of the desired protein in a cell. Together, the nucleic acid encoding the desired protein and its promoter/regulatory sequence comprise a “transgene.”

“Inducible” expression is a state in which a gene product is produced in a living cell in response to the presence of a signal in the cell.

A “recombinant polypeptide” is one, which is produced upon expression of a recombinant polynucleotide.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

As used herein, the term “transgenic mammal” means a mammal, the germ cells of which, comprise an exogenous nucleic acid.

As used herein, to “treat” means reducing the frequency with which symptoms of the inflammatory disease, are experienced by a patient, or altering the natural history and/or progression of the disease in a patient.

As used herein, the term “antisense oligonucleotide” means a nucleic acid polymer, at least a portion of which is complementary to a nucleic acid which is present in a normal cell or in an affected cell. Most preferably, the antisense oligonucleotides comprise between about fifteen and about fifty nucleotides. The antisense oligonucleotides of the invention include, but are not limited to, phosphorothioate oligonucleotides and other modifications of oligonucleotides.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

A “portion” of a polynucleotide means at least at least about fifteen to about fifty sequential nucleotide residues of the polynucleotide. It is understood that a portion of a polynucleotide may include every nucleotide residue of the polynucleotide.

By the term “specifically binds,” as used herein, is meant an antibody which recognizes and binds VEGF or its receptor, but does not substantially recognize or bind other molecules in a sample.

A “prophylactic” treatment is a treatment administered to a subject who does not exhibit signs of a disease or exhibits only early signs of the disease for the purpose of decreasing the risk of developing pathology associated with the disease.

“Preventing” a disease, as the term is used herein, means that the onset of the disease is delayed, and/or that the symptoms of the disease will be decreased in intensity and/or frequency, when a VEGF antagonist is administered compared with the onset and/or symptoms in the absence of the antagonist.

A “therapeutic” treatment is a treatment administered to a subject who exhibits signs of pathology for the purpose of diminishing or eliminating those signs.

As used herein, the term “neovascularization” refers to the growth of blood vessels and capillaries.

As used herein, “inhibit”, “inhibiting” or “inhibition” includes any measurable reproducible reduction in the interaction of VEGF and KDR or any other activities VEGF may mediate.

Methods:

A. Methods of Treating an Inflammatory Disease

The present invention includes a method of treating a Th2-mediated inflammatory disease wherein the disease is associated with an increased level of VEGF. Contemplated in the present invention are methods of treating the inflammatory disease in a mammal, preferably a human, using a VEGF antagonist. This is because, as would be appreciated by one skilled in the art when provided with the disclosure herein, inhibiting the expression and/or activity of VEGF serves as a treatment for inflammatory diseases. That is, the data disclosed herein demonstrate VEGF induces asthma-like phenotype with inflammation, parenchymal and vascular remodeling, edema, mucus metaplasia, myocyte hyperplasia, and airways hyperresponsiveness. The mucus response is mediated by an IL-13-dependent and the rest of the asthma-like phenotype is mediated by an IL-13-independent pathway(s). VEGF also enhanced respiratory antigen sensitization and Th2 inflammation and increased the number of activated DC2 dendritic cells. Thus, the present invention relates to treating of such diseases using VEGF antagonists, including, but not limited to, a VEGF antagonist (e.g., SU1498).

It would be understood by one skilled in the art, based upon the disclosure provided herein, that partial or complete inhibition of VEGF encompasses inhibition of VEGF expression, such as that mediated by, among other things, a ribozyme, siRNA, and/or antisense molecule that inhibits expression of a nucleic acid encoding VEGF. Additionally, the skilled artisan would appreciate, once armed with the teachings of the present invention, that inhibition of VEGF includes inhibition of VEGF activity in a cell. Such inhibition of VEGF activity can be effected using antagonists of VEGF itself including, inter alia, VEGF_{R1R2} Trap or the VEGF receptors including, inter alia, SU5614 and SU1498. Further, antagonists of VEGF activity include an antibody that specifically binds with VEGF thereby preventing the protein from functioning. Thus, a VEGF antagonist includes, but is not limited to, inhibiting transcription, translation, or both, of a nucleic acid encoding VEGF; and it also includes inhibiting any activity of the protein as well.

The present invention includes a method of treating or preventing a Th2 mediated inflammatory disease in a mammal. The method comprises administering a VEGF antagonist to a mammal in need of such treatment. This is because, as would be appreciated by one skilled in the art armed with the teachings of the present invention, inhibiting VEGF is useful for treating or preventing a Th-2 mediated inflammatory disease. Partial or complete inhibition of VEGF prevents, in turn, the pathology associated with a Th2 inflammatory disease, as amply demonstrated by the data disclosed herein.

More specifically, the invention relates to reducing or inhibiting VEGF using various antagonists. That is, one skilled in the art would understand, based upon the disclosure provided herein, that compounds that inhibit the expression, activity, and/or function of VEGF encompass, but are not limited to, an antibody, a VEGF trap an antisense nucleic acid, a ribozyme, a small molecule, a peptidomimetic and an interfering RNA molecule, either known or to be developed, which inhibits VEGF, and thereby a Th2 mediated inflammatory disease.

One skilled in the art would appreciate, based on the disclosure provided herein, that an antagonist of the invention includes molecules and compounds that prevent or inhibit the expression, activity or function of VEGF in a mammal. That is, the invention contemplates that an antisense and/or antisense molecule that inhibits, decreases, and/or abolishes expression of VEGF such that the VEGF is not detectable in the cell or tissue is an antagonist of the invention. For instance, a compound that degrades VEGF can decrease its function, and can be an antagonist as contemplated in the present invention.

Inhibition of VEGF can be assessed using a wide variety of methods, including those disclosed herein, as well as methods well-mown in the art or to be developed in the future. That is, the routineer would appreciate, based upon the disclosure provided herein, that inhibition of VEGF expression can be readily assessed using methods that assess the level of a nucleic acid encoding VEGF (e.g., mRNA) and/or the level of VEGF present in a cell or fluid.

One skilled in the art, based upon the disclosure provided herein, would understand that the invention encompasses treatment of a variety of Th2 mediated/associated inflammatory diseases, including, but not limited to, asthma, chronic obstructive pulmonary disease, interstitial lung disease, chronic obstructive lung disease, chronic bronchitis, eosinophilic bronchitis, eosinophilic pneumonia, pneumonia, inflammatory bowel disease, atopic dermatitis, atopy, allergy, allergic rhinitis, idiopathic pulmonary fibrosis, scleroderma, and the like. As disclosed herein, these diseases involve and/or are mediated by, increased VEGF in tissues where increased VEGF includes, and is not limited to, increased VEGF expression, increased VEGF activity, or both.

Further, the skilled artisan would further appreciate, based upon the teachings provided herein, that the diseases encompass any disease comprising increased VEGF in a tissue including, among others, a disease mediated by increased IL-13 production. This is because, as more fully set forth elsewhere herein, the data disclosed herein demonstrate that overexpression of VEGF mediates an increased expression of IL-13 mRNA which, in turns, mediates and/or is associated with a variety of changes associated with inflammatory disease including, but not limited to, tissue inflammation and increased mucus metaplasia.

Therefore, the data disclosed herein demonstrate that inhibition of VEGF in a mammal afflicted with an inflammatory disease, wherein the disease is mediated or associated with increased expression of IL-13, will treat the disease by mediating a decrease in the level of VEGF which, in turn, treats the disease. For instance, such data include, but are not limited to, the inhibition of various tissue pathology by administering a VEGF antagonist (e.g., SU 1498) to a mammal where increased expression of VEGF mediates increased expression of IL-13.

The present invention further comprises a method for treating a Th2 mediated inflammatory disease mediated by and/or associated with a Th2 inflammatory response in a mammal. The skilled artisan, when armed with the present disclosure and the teachings provided herein, would understand that a Th2 mediated inflammatory disease mediated by and/or associated with a Th2 inflammatory response encompasses a variety of inflammatory diseases, including, but not limited to, asthma, chronic obstructive pulmonary disease, interstitial lung disease, chronic obstructive lung disease, chronic bronchitis, eosinophilic bronchitis, eosinophilic pneumonia, pneumonia, inflammatory bowel disease, atopic dermatitis, atopy, allergy, allergic rhinitis, idiopathic pulmonary fibrosis, scleroderma, and the like. As disclosed herein, these diseases are mediated by a Th2 inflammatory response in an mammal, and may result in, among other things, increased IL-13 production and/or expression, increased VEGF activity and/or expression, and the like.

Further, the skilled artisan would appreciate, based upon the teachings provided herein, that the diseases encompass any disease comprising increased VEGF in a tissue including, among others, a disease mediated by increased Th2 inflammatory response. This is because, as more fully set forth elsewhere herein, the data disclosed herein demonstrate that increased Th2 inflammatory responses may result in, inter alia, increased IL-13 activity and/or expression which, in turns, mediates and/or is associated with a variety of changes associated with inflammatory diseases

Therefore, the data disclosed herein demonstrate that inhibition of VEGF in a mammal afflicted with an inflammatory disease, wherein the disease is mediated by and/or associated with an increased Th2 inflammatory response, will treat the disease by mediating a decrease in the level of VEGF which, in turn, treats the disease. For instance, such data include, but are not limited to, the inhibition of various tissue pathology by administering a VEGF antagonist (e.g., SU1498) to a mammal where a Th2 inflammatory response mediates increased VEGF activity and increased VEGF expression.

A VEGF antagonist can include, but should not be construed as being limited to a biological molecule and a small molecule. Biological molecules include all lipids and polymers of monosaccharides, amino acids and nucleotides having a molecular weight greater than 450 Kd. Thus, biological molecules include, for example, oligosaccharides and polysaccharides; oligopeptides, polypeptides, peptides, peptidomimetic, and proteins; and oligonucleotides and polynucleotides. Oligonucleotides and polynucleotides include, for example, DNA and RNA such as a ribozyme or an antisense nucleic acid molecule.

Biological molecules further include derivatives of any of the molecules described above. For example, derivatives of biological molecules include lipid and glycosylation derivatives of oligopeptides, polypeptides, peptides and proteins. Derivatives of biological molecules further include lipid derivatives of oligosaccharides and polysaccharides, e.g. lipopolysaccharides.

Any molecule that is not a biological molecule is considered in this specification to be a small molecule. Some examples of small molecules include chemical compounds, organometallic compounds, salts of organic and organometallic compounds, saccharides, amino acids, nucleosides and nucleotides. Small molecules further include molecules that would otherwise be considered biological molecules, except their molecular weight is not greater than 450 Kd. Thus, small molecules may be lipids, oligosaccharides, oligopeptides, and oligonucleotides, and their derivatives, having a molecular weight of 450 Kd or less.

It is emphasized that small molecules can have any molecular weight. They are merely called small molecules because they typically have molecular weights less than 450 Kd. Small molecules include compounds that are found in nature as well as synthetic compounds. One of skill in the art would readily appreciate, based on the disclosure provided herein, that a VEGF antagonist encompasses a chemical compound that inhibits the activity of VEGF. VEGF antagonists are well known in the art, and some of the key critical elements of one class of VEGF antagonists have been defined (Underiner et al. al., 2004 Curr. Med. Chem. 6:731-45). Additionally, a VEGF antagonist encompasses a chemically modified compound, and derivatives, as is well known to one of skill in the chemical arts.

The skilled artisan would appreciate that a VEGF antagonist encompasses an already known VEGF antagonist such as, but not limited to, SU1498, Pyrrolo[2,3-d]pyrimidine nucleoside analogs (see, e.g., U.S. Pat. No. 6,831,069), heterocyclic substituted pyrazolones (see, e.g., U.S. Pat. No. 6,831,075), macroheterocyclic compounds (see, e.g., U.S. Pat. No. 6,828,327), anthranylalkyl and cycloalkyl amides (see, e.g., U.S. Pat. No. 6,818,661), 3-(anilinomethylene)oxindoles (see, e.g., U.S. Pat. No. 6,818,632), humanized neutralizing antibodies (Kim, et al., 1993 Nature 362: 841-844; Muller et al. 1997 Proc. Nat. Acal. Sci. 94:7192-7197; Muller et al., 1998 Structure 6:1153-1167; U.S. Pat. No. 5,855,866), SELEX-derived RNA molecules (Jellinek et al., (1994) Biochemistry 33:10450-10456; U.S. Pat. No. 5,859,228), anti KDR monoclonal antibodies (Witte et al., 1998 Cancer & Metast. Reviews 17:155-161; U.S. Pat. No. 5,840,301), soluble Fit receptor (U.S. Pat. No. 5,861,484), fragments of VEGF (U.S. Pat. No. 5,240,848), anti VEGF antisense oligonucleotide (U.S. Pat. No. 5,641,756), peptide VEGF antagonists (U.S. Pat. No. 6,777,534), VEGF receptor antibody (U.S. Pat. No. 6,811,779).

Further, one of skill in the art would, when equipped with this disclosure and the methods exemplified herein, appreciate that a VEGF antagonist includes such antagonists as discovered in the future, as can be identified by well-known criteria in the art of pharmacology, such as the physiological results of inhibition of VEGF as described in detail herein and/or as known in the art. Therefore, the present invention is not limited in any way to any particular VEGF antagonist as exemplified or disclosed herein; rather, the invention encompasses those antagonists that would be understood by the routineer to be useful as are known in the art and as are discovered in the future.

Further methods of identifying and producing a VEGF antagonist are well known to those of ordinary skill in the art, including, but not limited, obtaining an antagonist from a naturally occurring source (i.e., Streptomyces sp., Pseudomonas sp., Stylotella aurantium). Alternatively, a VEGF antagonist can be synthesized chemically. Further, the routineer would appreciate, based upon the teachings provided herein, that a VEGF antagonist can be obtained from a recombinant organism. Compositions and methods for chemically synthesizing VEGF antagonists are well known in the art and are described herein.

The skilled artisan would also appreciate, based on the disclosure provided herein, that a VEGF antagonist encompasses an antibody that specifically binds with VEGF or its receptor, thereby inhibiting the action of these proteins. For instance, antibodies that specifically bind to VEGF receptors are well known to those of ordinary skill in the art (see, e.g., U.S. Pat. No. 6,811,779). Similarly, antibodies to VEGF receptor can be produced using standard methods disclosed herein or well known to those of ordinary skill in the art (Harlow et al., 1988, Antibodies: A Laboratory Manual, Cold Spring Harbor, N.Y.). Thus, the present invention is not limited in any way to any particular antibody; instead, the invention includes any antibody that specifically binds with VEGF or a VEGF receptor either known in the art and/or identified in the future.

One of skill in the art will appreciate that an antibody can be administered as a protein, a nucleic acid construct encoding a protein, or both. Numerous vectors and other compositions and methods are well known for administering a protein or a nucleic acid construct encoding a protein to cells or tissues. Therefore, the invention includes a method of administering an antibody or nucleic acid encoding an antibody (e.g., synthetic antibody) that is specific for VEGF or its receptor. (Sambrook et al., 1989, Molecular Cloning: A Laboratory Manual, Cold Spring Harbor Laboratory, New York; Ausubel et al., 1997, Current Protocols in Molecular Biology, John Wiley & Sons, New York).

The skilled artisan would understand, based upon the disclosure provided herein, that the invention encompasses administering an antibody that specifically binds with VEGF or its receptor of interest, or a nucleic acid encoding the antibody, wherein the antibody molecule further comprises an intracellular retention sequence such that the antibody binds with the VEGF receptor and prevents its expression at the cell surface and/or its export from a cell. Such antibodies, frequently referred to as “intrabodies”, are well known in the art and are described in, for example, Marasco et al. (U.S. Pat. No. 6,004,490) and Beerli et al. (1996, Breast Cancer Research and Treatment 38:11-17).

The skilled artisan would also appreciate, based on the disclosure provided herein, that a VEGF antagonist encompasses a VEGF trap that specifically binds with VEGF, thereby inhibiting the action of the protein. VEGF traps are fusions between VEGF receptor components and the Fc portion of IgG. VEGF traps are a novel extension of the receptor-Fc fusion concept in that they include two distinct receptor components that bind a single VEGF molecule, resulting in the generation of blockers with dramatically increased affinity over that offered by single component reagents. For example, the VEGF Trap_{R1R2} disclosed herein is a fusion protein comprised of the extracellular domains of VEGF_{R1} and VEGF_{R2} coupled to the Fc-portion of IgG that binds VEGF-A, but not VEGF-B or VEGF-C. VEGF traps can also be generated by employing components of other VEGF receptor subtypes. Thus, the present invention is not limited in any way to any particular VEGF traps; instead, the invention includes any VEGF traps that specifically binds with VEGF or VEGF isomers.

The present invention is not limited to chemical compounds, antibodies or VEGF traps against VEGF. One of skill in the art would appreciate that inhibiting the expression of a polypeptide is likewise an effective method of inhibiting the activity and function of the polypeptide. Thus, a method is provided for the inhibition of VEGF by inhibiting the expression of a nucleic acid encoding VEGF. Methods to inhibit the expression of a gene are well known to those of ordinary skill in the art, and include the use of ribozymes or antisense oligonucleotide.

Antisense oligonucleotides are DNA or RNA molecules that are complementary to some portion of an mRNA molecule. When present in a cell, antisense oligonucleotides hybridize to an existing mRNA molecule and inhibit translation into a gene product. Inhibiting the expression of a gene using an antisense oligonucleotide is well known in the art (Marcus-Sekura, 1988, Anal. Biochem. 172:289), as are methods of expressing an antisense oligonucleotide in a cell (Inoue, U.S. Pat. No. 5,190,931).

Contemplated in the present invention are antisense oligonucleotides that are synthesized and provided to the cell by way of methods well known to those of ordinary skill in the art. As an example, an antisense oligonucleotide can be synthesized to be between about 10 and about 100, more preferably between about 15 and about 50 nucleotides long. The synthesis of nucleic acid molecules is well known in the art, as is the synthesis of modified antisense oligonucleotides to improve biological activity in comparison to unmodified antisense oligonucleotides (Tullis, 1991, U.S. Pat. No. 5,023,243).

Similarly, the expression of a gene may be inhibited by the hybridization of an antisense molecule to a promoter or other regulatory element of a gene, thereby affecting the transcription of the gene. Methods for the identification of a promoter or other regulatory element that interacts with a gene of interest are well known in the art, and include such methods as the yeast two hybrid system (Bartel and Fields, eds., In: The Yeast Two Hybrid System, Oxford University Press, Cary, N.C.).

Alternatively, inhibition of a gene expressing VEGF can be accomplished through the use of a ribozyme. Using ribozymes for inhibiting gene expression is well known to those of skill in the art (see, e.g., Cech et al., 1992, J. Biol. Chem. 267:17479; Hampel et al., 1989, Biochemistry 28: 4929; Altman et al., U.S. Pat. No. 5,168,053). Ribozymes are catalytic RNA molecules with the ability to cleave other single-stranded RNA molecules. Ribozymes are known to be sequence specific, and can therefore be modified to recognize a specific nucleotide sequence (Cech, 1988, J. Amer. Med. Assn. 260:3030), allowing the selective cleavage of specific mRNA molecules. Given the nucleotide sequence VEGF, one of ordinary skill in the art could synthesize an antisense oligonucleotide or ribozyme without undue experimentation, provided with the disclosure and references incorporated herein.

Further, inhibition of a gene expressing VEGF can be achieved through the use of interfering RNA. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., Nature (1998) 391(19):306-311; Timmons et al., Nature (1998) 395:854; Montgomery et al., TIG (1998) 14(7):255-258; David R. Engelke, Ed., RNA Interference (RNAi) Nuts & Bolts of RNAi Technology, DNA Press (2003); and Gregory J. Hannon, Ed., RNAi A Guide to Gene Silencing, Cold Spring Harbor Laboratory Press (2003). Therefore, the present invention also includes methods of silencing the gene encoding VEGF by using RNAi technology.

One of skill in the art will appreciate that antagonists of VEGF gene expression can be administered singly or in any combination thereof. Further, VEGF antagonists can be adminstered singly or in any combination thereof in a temporal sense, in that they may be administered simultaneously, before, and/or after each other. One of ordinary skill in the art will appreciate, based on the disclosure provided herein, that VEGF antagonists to inhibit gene expression can be used to treat asthma and other Th2 mediated inflammatory diseases and that an antagonist can be used alone or in any combination with another antagonist to effect a therapeutic result.

B. Method of Preventing an Inflammatory Disease

It will be appreciated by one of skill in the art, when armed with the present disclosure including the methods detailed herein, that the invention is not limited to treatment of a Th2 mediated inflammatory disease once the disease is established. Particularly, the symptoms of the disease need not have manifested to the point of detriment to the mammal; indeed, the disease need not be detected in a mammal before treatment is administered. That is, significant pathology from an inflammatory disease does not have to occur before the present invention may provide benefit. Therefore, the present invention, as described more fully herein, includes a method for reducing or preventing an inflammatory disease in a mammal, in that a VEGF antagonist, as discussed previously elsewhere herein, can be administered to a mammal prior to the onset of a Th2 inflammatory disease, thereby preventing the disease as demonstrated by the data disclosed herein.

One of skill in the art, when armed with the disclosure herein, would appreciate that the reduction or prevention of Th2 mediated inflammatory disease encompasses administering to a mammal a VEGF antagonist as a preventative measure against inflammatory disease. As detailed herein, the symptoms and etiologies of VEGF-associated inflammatory disease include tissue inflammation, parenchymal and vascular remodeling, edema, mucus metaplasia, myocyte hyperplasia, and airways hyperresponsiveness. Given these etiologies and the methods disclosed elsewhere herein, the skilled artisan can recognize and prevent a Th2 mediated inflammatory disease in a mammal using a VEGF antagonist before the disease pathology can be detected.

This is because the data disclosed herein demonstrate that administration of a VEGF antagonist, including, but not limited to, SU1498 or VEGF_{R1R2} Trap, prevented onset of a Th2 mediated inflammatory disease in a mammal, whether the disease was induced by an allergen (e.g. ovalbumin sensitization) or whether the mammal was genetically predisposed to the disease (e.g., transgenic mice inducibly overproducing VEGF). Accordingly, the skilled artisan would appreciate, based on the disclosure provided elsewhere herein, that the present invention includes a method of preventing disease comprising inhibiting VEGF using a VEGF antagonist. Further, as more fully discussed elsewhere herein, methods of inhibiting VEGF encompass a wide plethora of techniques for inhibiting not only VEGF activity, but also for inhibiting expression of a nucleic acid encoding VEGF. Additionally, as disclosed elsewhere herein, one skilled in the art would understand, once armed with the teaching provided herein, that the present invention encompasses a method of preventing a wide variety of diseases where expression and/or activity of VEGF mediates the disease. Methods for assessing whether a disease relates to over expression or increased activity of VEGF are disclosed elsewhere herein and/or are well-known in the art. Further, the invention encompasses treatment or prevention of such diseases discovered in the future.

The invention encompasses administration of a VEGF antagonist to practice the methods of the invention; the skilled artisan would understand, based on the disclosure provided herein, how to formulate and administer the appropriate VEGF antagonist to a mammal. Indeed, the successful administration of VEGF antagonists have been extensively reduced to practice as exemplified herein. However, the present invention is not limited to any particular method of administration or treatment regimen. This is especially true where it would be appreciated by one skilled in the art, equipped with the disclosure provided herein, including the extensive reduction to practice using an art-recognized model of inflammatory disease, that methods of administering a VEGF antagonist can be readily determined by one of skill in the pharmacological arts.

As used herein, the term “pharmaceutically-acceptable carrier” means a chemical composition with which an appropriate VEGF antagonist may be combined and which, following the combination, can be used to administer the appropriate VEGF antagonist to a mammal.

The pharmaceutical compositions useful for practicing the invention may be administered to deliver a dose of between about 0.1 ng/kg/day and 100 mg/kg/day.

Pharmaceutical compositions that are useful in the methods of the invention may be administered systemically in oral solid formulations, ophthalmic, suppository, aerosol, topical or other similar formulations. In addition to the appropriate VEGF antagonist, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer an appropriate VEGF antagonist according to the methods of the invention.

Compounds which are identified using any method described herein as potential useful compounds for treatment and/or prevention of a disease of interest can be formulated and administered to a mammal for treatment of the diseases disclosed herein are now described.

The invention encompasses the preparation and use of pharmaceutical compositions comprising a compound useful for treatment of the diseases disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats and dogs, and birds including commercially relevant birds such as chickens, ducks, geese, and turkeys.

Pharmaceutical compositions that are useful in the methods of the invention may be prepared, packaged, or sold in formulations suitable for oral, rectal, vaginal, parenteral, topical, pulmonary, intranasal, buccal, intravenous, ophthalmic, intrathecal or another route of administration. Other contemplated formulations include projected nanop articles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an allylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e. about 20° C.) and which is liquid at the rectal temperature of the subject (i.e. about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or gel or cream or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject. Douche preparations may further comprise various additional ingredients including, but not limited to, antioxidants, antibiotics, antifungal agents, and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intravenous, intramuscular, intracisternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, contain 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically dosages of the compound of the invention which may be administered to an animal, preferably a human, range in amount from about 0.01 mg to about 100 g per kilogram of body weight of the animal. While the precise dosage administered will vary depending upon any number of factors, including but not limited to, the type of animal and type of disease state being treated, the age of the animal and the route of administration. Preferably, the dosage of the compound will vary from about 1 mg to about 100 mg per kilogram of body weight of the animal. More preferably, the dosage will vary from about 1 μg to about 1 g per kilogram of body weight of the animal. The compound can be administered to an animal as frequently as several times daily, or it can be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

C. Methods of Identifying a Useful Compound

The invention encompasses a method for identifying a compound or intervention that treats a Th2 mediated inflammatory disease. One skilled in the art would appreciate, based upon the disclosure provided herein, that assessing the expression and/or activity of VEGF can be performed by assessing, among other things, the levels of VEGF or the mRNA that encodes it in a cell or tissue, and the like, and then the level can be compared to the level in an otherwise identical cell or tissue to which the compound is not administered. Alternatively, the level of VEGF or the mRNA that encode it in a cell or tissue contacted with a compound can be compared with the level of the VEGF or its mRNA in the cell or tissue prior to administration of the compound. One skilled in the art would understand that such compound can be a useful potential therapeutic for treating and/or preventing a Th2 mediated inflammatory disease, and/or for treating a disease associated with and/or mediated by a Th2 inflammatory response.

The skilled artisan would further appreciate that the methods for identifying a compound useful for inhibiting VEGF include methods wherein a compound is administered to a cell, tissue, or animal. That is, the skilled artisan, when armed with the present disclosure, would recognize that the teachings herein can be used to identify a compound useful for inhibiting VEGF in a cell or tissue expressing VEGF. Such cells and tissues are well known in the art, and can include cells and tissues derived from a transgenic non-human animal having altered expression of VEGF, or a transgenic animal comprising an inflammatory disease, and/or a cell or tissue derived therefrom.

Additionally, a cell or tissue comprising expression of VEGF can be contacted with a compound and the level of the VEGF can be assessed and compared to the level of the VEGF in the cell and/or tissue prior to administration of the compound. Further, the level of the VEGF can be compared to the level of the VEGF in an otherwise identical cell or tissue not contacted with the compound.

One skilled in the art would appreciate, based upon the disclosure provided herein, that the cell or tissue can express endogenous VEGF, but the invention further encompasses a cell or tissue that has been modified to express VEGF not otherwise expressed in the tissue, e.g., a nucleic encoding VEGF of interest can be introduced and expressed in the cell or tissue where it is not typically expressed, or is expressed at a different level than after the nucleic acid is introduced into the cell or tissue. Thus, the invention includes a wide plethora of assays, comprising a cell, tissue, or an animal, wherein the level of VEGF can be assessed in the presence or absence of a compound. Accordingly, the skilled artisan would be able to identify a compound using the methods disclosed herein and cell culture and cell propagation techniques well known in the art to assess the ability of a compound to affect the level of VEGF. Therefore, the present invention further encompasses a method of identifying a compound useful for inhibiting VEGF in a cell or tissue, as well as in an animal.

One of skill in the art would understand, based upon the disclosure provided herein, that the invention includes a method of identifying a compound useful for treating a Th2 mediated inflammatory disease in a mammal. As would be understood by one skilled in the art armed with the teachings provided herein, the method encompasses identifying a compound that treats an inflammatory disease in a cell or tissue. The method comprises identifying a substance or compound that inhibits the expression and/or activity of VEGF in a mammal (including in a cell or tissue thereof), preferably in the respiratory tract. This is because, as discussed elsewhere herein, the data demonstrate that inhibiting the expression or activity of VEGF provides a therapeutic benefit thereby treating or preventing an inflammatory disease mediated by or associated with increased expression or activity of VEGF. This is because the present invention discloses, for the first time, that increased level of VEGF is associated with, or mediates, such disease and that inhibiting the VEGF, using a VEGF antagonist (e.g., SU1498, a synthetic antibody specific for the VEGF, such as, but not limited to, a VEGF_{R1R2} Trap) prevents and/or treats the disease.

Thus, the skilled artisan, once armed with the teachings of the invention, would appreciate that a compound that inhibits VEGF is a powerful potential therapeutic or prophylactic treatment of inflammatory disease, such that identification of such a compound identifies a potential therapeutic for such disease.

The method comprises administering to a mammal afflicted with an inflammatory disease, a compound, and comparing the level of VEGF in the mammal before and after administration of the compound. The routineer would understand, based on the disclosure provided herein, that a lower level of VEGF or the mRNA that encodes it in the mammal after administration of the compound compared with the level of VEGF or its mRNA before administration of the compound indicates that the compound is useful for treating a Th2 mediated inflammatory disease in a mammal.

This is because, as stated previously elsewhere herein, it has been discovered that inhibiting VEGF in an animal treats or prevents a disease associated with increased VEGF expression and/or activity, e.g., a Th2 mediated inflammatory disease with enhanced tissue remodeling and AHR. The skilled artisan would also appreciate, in view of the disclosure provided herein, that assays to determine the level of VEGF in a mammal, including a cell or tissue thereof, include those well known in the art, or those to be developed in the future, all of which can be used to assess the level of VEGF in a mammal (or cell or tissue thereof) before and after administration of the compound The skilled artisan would further appreciate that the levels of VEGF, as disclosed elsewhere herein, include levels of VEGF activity and levels of VEGF expression. Further, the invention encompasses a compound identified using this method.

The invention further includes additional methods for identifying a compound useful for inhibiting VEGF and thereby a Th2 mediated inflammatory disease in a mammal. More specifically, the method comprises assessing the level of VEGF expression, production, or activity in a mammal (or a cell or tissue thereof) to which the compound is administered in comparison to an identical mammal (or cell or tissue thereof) to which the compound is not administered. Additionally, the method comprises comparing the level of VEGF in the same mammal, or cell or tissue thereof, before and after administration of a compound of interest. A lower level of VEGF expression, production, or activity in the mammal administered the compound when compared to an identical mammal not administered the compound, or to the same mammal prior to administration of the compound, is an indication that the compound is useful for inhibiting VEGF which is therefore a useful potential therapeutic to treat and/or prevent inflammatory disease in a mammal. This is because the present invention discloses, for the first time, that VEGF plays a clear role in the pathology of Th2 mediated inflammatory diseases and that inhibiting VEGF treats and/or prevents disease in an art-recognized animal model of inflammatory disease. Clearly, as demonstrated elsewhere herein, a compound that inhibits VEGF is an important potential therapeutic compound useful for treatment and prevention of inflammatory disease as demonstrated by the data disclosed herein.

The methods detailed above include mammals in which the levels of VEGF can be readily assessed using the methods described herein. Thereby, the present invention includes mammals useful for identifying a compound that can be used for the treatment or prevention of inflammatory diseases. More particularly, the invention includes transgenic animals inducible expressing VEGF in the respiratory tract. Based on the disclosure provided herein, such transgenic mammals, when administered a compound, can be readily assayed for levels of VEGF, whether the assay be for VEGF expression or VEGF activity. And such methods of identifying a compound useful for treating and/or preventing a Th2 mediated inflammatory disease relating to using of transgenic non-human mammals to assess whether the compound inhibits VEGF are encompassed in the present invention. The methods detailed above are also applicable to methods of identification of a compound through assays of genes and gene products that are known downstream of VEGF.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### EXPERIMENTAL EXAMPLES

The invention is now described with reference to the following examples. These examples are provided for the purpose of illustration only and the invention should in no way be construed as being limited to these examples but rather should be construed to encompass any and all variations which become evident as a result of the teaching provided herein.

The materials and methods used in the experiments presented in this Example are now described.

### Example 1

### Generation of Inducible VEGF Transgenic Mice

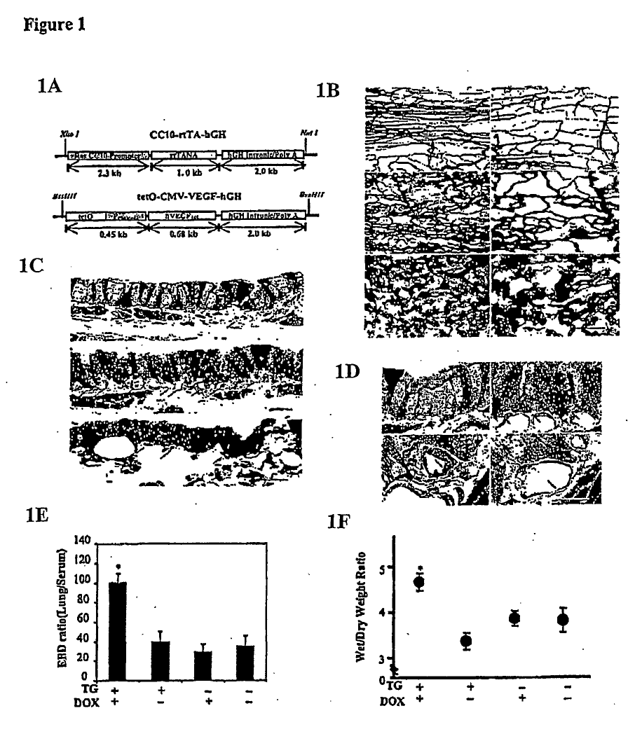

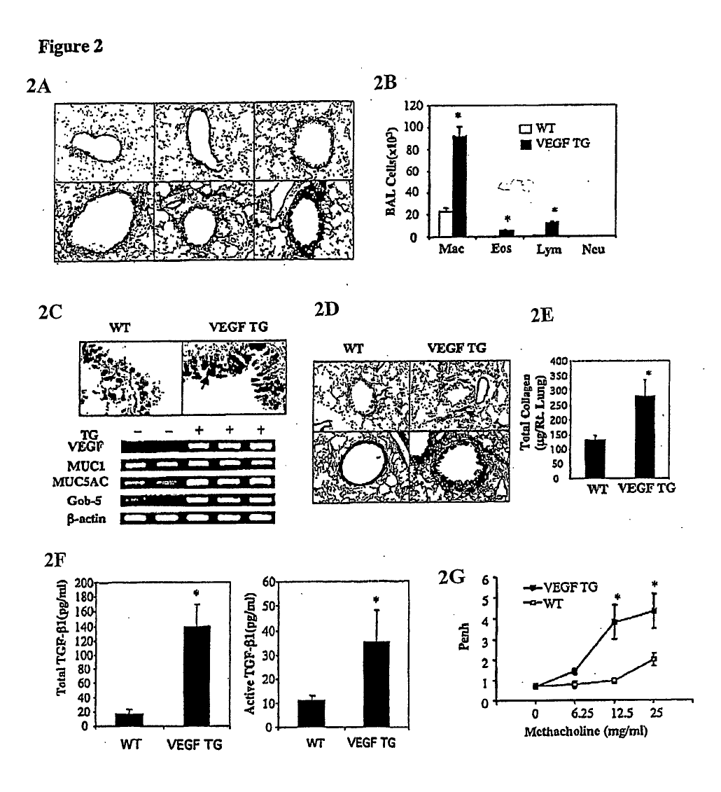

The CC10-rtTA-VEGF transgenic mice in these studies used the Clara cell 10-kDa protein (CC10) promoter and two transgenic constructs to target VEGF to the lung in an externally regulatable fashion. These mice were generated using the transgenic system and approaches previously described in Zheng et al. and Ray et al. (Zheng et al. 2000, J. Clin. Invest. 106:1081-1093; Ray et al. 1997, J. Clin. Invest. 100:2501-2511). Construct 1, CC10-rtTA-hGH, contains the CC10 promoter, the reverse tetracycline transactivator (rtTA) and human growth hormone (hGH) intronic and polyadenylation sequences (FIG. 1A). Construct 2, tet-O-CMV-VEGF-hGH, contains a polymeric tetracycline operator (tet-O), minimal cytomegalovirus (CMV) promoter, human VEGF₁₆₅ cDNA and hGH (FIG. 1A). This construct was prepared by replacing the IL-11 cDNA in construct tet-O-CMV-hIL-11 described in Ray et al (Ray et al. 1997, J. Clin. Invest. 100:2501-2511) with human VEGF₁₆₅.

All animals were evaluated for the presence of both rtTA and VEGF₁₆₅ using PCR analysis. rtTA was evaluated as described in Zheng et al. (Zheng et al. 2000, J. Clin. Invest. 106:1081-1093). PCR for VEGF₁₆₅ was undertaken using the following primers; sense CCTCCGCGGCCATGAACTTT (SEQ ID NO: 1) and antisense TCTTTCCGGATCCGAGATCTGG (SEQ ID NO: 2). All founder animals were bred for at least 8 generations onto a C57BL/6 background.

One month old TG and WT littermate controls were randomized to normal water or water containing doxycycline (dox) (0.5 mg/ml) as described (Zheng et al., 2000) and evaluated at intervals thereafter. BAL VEGF quantification, histologic analysis, mRNA analysis, in situ hybridization, immunohistochemistry (IHC), collagen quantification, and the quantification of total and bioactive TGF-β₁ were undertaken as described (Zheng et al., 2000; Ray et al., 1997; Corne et al., 2000; Lee et al., 2001, 2002). Airway methacholine responsiveness was assessed using non-invasive whole body phlethysmography as described (Zhu et al., 1999; Park et al., 2001).

### Statistics

Data that were normally distributed were assessed using the Student's T test or ANOVA and data that were not normally distributed were assessed using the Wilcoxon rank sum test as appropriate.

### Breeding to IL-13 Null Mutant (−/−) Mice

CC10-rtTA-VEGF animals were bred with WT animals and IL-13^{-/-} animals (from Professor. A. N. J. McKenzie, Cambridge, U.K.) as previously described (Lee et al., 2002).

### Results:

VEGF overexpression during lung development causes fetal lethality (Zeng et al., 1998). To avoid this issue, constructs in FIG. 1A in the externally regulatable dual construct transgenic system developed were used (Zheng et al., 2000; Ray et al., 1997). Three dual transgene (+) (TG) CC10-rtTA-VEGF founder mice were identified. At 1 month of age they were randomized to normal water or doxycycline (dox) water. In the wild type (WT) mice on normal or dox water and the TG mice on normal water, VEGF levels in bronchioalveolar lavage fluids (BAL) were ≦10 pg/ml. Increased levels of BAL VEGF were noted within 24 hours and steady state levels between 0.05 and 12 ng/ml were seen after 1 week of dox administration (data not shown). Interestingly, these levels of VEGF are in accord with the levels in biologic fluids from normals and patients with asthma or respiratory syncytial virus infection (Kanazawa et al., 2002; Asai et al., 2002; Lee et al., 2000; Ohta et al., 2002; Meyer et al., 2000; Nishigaki et al., 2003). In all cases, RT-PCR evaluations of multiple organs and tissues demonstrated that VEGF was produced in a lung-specific fashion (data not shown).

### Example 2

### Effect on Neovascularization and Edema

### Electron Microscopy (EM)

After vascular perfusion with a fixative containing 1% paraformaldehyde and 3% glutaraldehyde in 100 mM sodium cacodylate buffer, pH 7.4, tissues were fixed overnight, sectioned, treated with 1% osmium tetroxide in 100 mM cacodylate buffer (pH 7.2) for 2 hr and then with 2% aqueous uranyl acetate for 48 hr, dehydrated in acetone, and embedded in epoxy. 0.5 μm sections were stained with toluidine blue for light microscopy, and 50-100 nM sections were stained with 0.8% lead citrate in 0.2 N NaOH for electron microscopy.

### Calculation of Wet/Dry Ratios

Lungs from dox and normal water treated mice were excised en bloc and extra-pulmonary tissues were removed. They were then immediately weighed and placed in a desiccating oven at 65° C. for 48 hours. After this incubation, dry weight was assessed, and the wet to dry ratio was calculated to quantify lung water content.

### Evans Blue Dye Extravasation

Evans blue dye (EBD) was used to assess plasma leakage as previously described (Kaner et al., 2000). The results are expressed as the ratio of the EBD absorbance at 620 nM of paired lung homogenates and serum.

### Staining of Airway Microvasculature

The vasculature was labeled by perfusion with a lectin that binds uniformly to the luminal surface of endothelial cells and intravascular leukocytes, as described previously (Thurston et al., 1996; Baluk et al., 1998).

### Results:

L. esculentum lectin analysis demonstrated that blood vessels in the tracheas and intrapulmonary bronchi of WT mice on normal or dox water and TG mice on normal water were arranged in orderly cascades with capillaries crossing areas between arterioles and venules (FIGS. 1B and 1C). In the mouse the bronchial circulation extended well into the mainstem extrapulmonary and intrapulmonary bronchi, but was less dense than in the trachea and gradually became sparser along the length of the bronchi (FIG. 1B, upper panels). In the TG animals, as little as 3 days of dox generated endothelial sprouts, mostly arising from the venules (FIG. 1B, middle panels). Vascular density (the % of the airway covered with vessels) was maximal after 7 days of dox and remained elevated for at least a month thereafter. At this time point, the vascular density in airways of TG mice was nearly twice that of WT mice (42.3±5.4 vs. 23.5±1.3%; P<0.01) (FIG. 1C). These new vessels were larger than the capillaries of the control airways (11.86±0.37 versus 8.90±0.14 μm, P<0.001) and, as seen in asthma (Vrugt et al., 2000), had migrated into the lamina reticularis and occasionally into the epithelium (FIGS. 1C and 1D). The endothelial cells of these new vessels were thin, had occasional fenestrations and were enveloped by pericyte processes and basement membranes. In these TG mice on dox water there was a significant increase in lung wet/dry ratios, Evans blue dye extravesation and bronchovascular edema when compared to the WT and TG controls (FIGS. 1E and F and data not shown) (P<0.01 comparing TG mice on dox water to all other groups). Thus, VEGF is a potent inducer of angiogenesis and edema in the murine airway and lung.

### Example 3

### Histologic and Physiologic Evaluation

Lungs of WT mice on normal or dox water and TG (+) mice on normal water did not show any abnormalities and could not be distinguished from lungs from TG mice on normal water (FIG. 2 and data not shown). In contrast, VEGF overexpression in the TG mice caused conspicuous alterations that persisted throughout the 5 month study interval. Inflammation was seen after 2 days of dox and persisted throughout the study. At early time points, an increase in tissue mononuclear cells, B lymphocytes and occasional clusters of eosinophils were noted (FIG. 2A and data not shown). At later time points, CD4+ and CD8+ T cells were also increased (1.82%±0.3 to 9.92±0.9% for CD4± cells, 1.14±0.4 to 8.64±0.7% for CD8+ cells, P<0.01 comparing WT to TG for both parameters). Similarly, total cell recovery and the recovery of macrophages, lymphocytes and eosinophils were increased in BAL from dox-treated TG mice (FIG. 2B). Mucus metaplasia was seen after 7 days of dox treatment. This response was characterized by D-PAS and Alcian blue staining airway epithelial cells, increased MUC5AC gene expression and increased epithelial gob-5 expression (FIG. 2C and data not shown). Myocyte hyperplasia with enlarged airway smooth muscle bundles was also seen after 7 days of dox administration (FIG. 2D). Histologically and biochemically detectable increases in collagen were first seen after 2 months and were most prominent after 4 months of VEGF overexpression (FIGS. 2D-2E). At these time points, VEGF also stimulated the production and spontaneous activation of TGF-β, (FIG. 2F). Thus, VEGF is potent stimulator of airway inflammation and airway remodeling with mucus metaplasia, subepithelial fibrosis and smooth muscle hyperplasia.

To assess the physiologic consequences of these alterations, responsiveness to methacholine was also assessed. When compared to WT mice on normal or dox water and TG mice on normal water, TG mice on dox water for as few as 7 days manifest impressive airways hyperresponsiveness (AHR) (FIG. 2G and data not shown). Thus, VEGF-induces AHR when elaborated in the murine airway.

### Example 4

### Induction and Role of IL-13

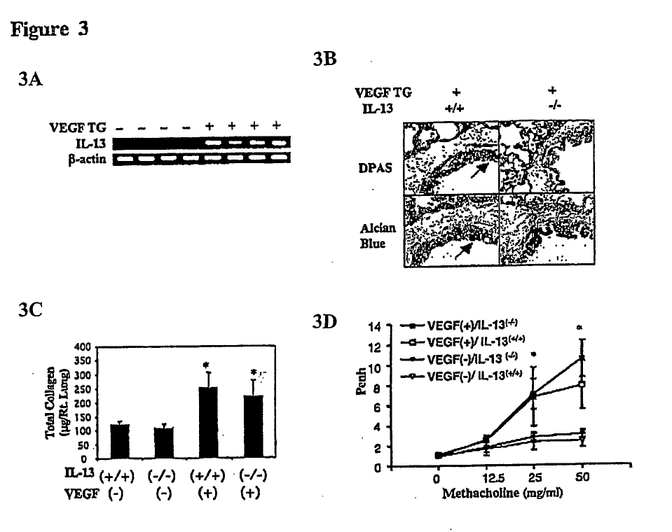

Studies were undertaken to determine if VEGF mediated its effects by stimulating Th2 cytokines. The levels of mRNA encoding IL-4, IL-5, and IL-9 in WT and TG mice on normal water or dox water were near or below the limits of detection of the assays (data not shown). Similarly, IL-13 mRNA could not be detected in RNA from WT mice on normal or dox water or TG mice on normal water (FIG. 3A and data not shown). In contrast, the lungs from TG mice on dox manifest increased expression of IL-13 mRNA (FIG. 3A).

To define the role of IL-13 in the VEGF phenotype, we compared the effects of VEGF in mice with (+/+) and (−/−) IL-13 loci. In the absence of IL-13, the ability of VEGF to induce mucus metaplasia was completely abrogated (FIG. 3B). In contrast, VEGF induction of tissue and BAL inflammation, neovascularization, AHR, myocyte hyperplasia, subepithelial fibrosis, TGF-β₁ elaboration and dendritic cell alterations (see below) were unaltered in IL-13^{-/-} animals (FIG. 3C-3D and data not shown). These studies document the IL-13-dependence of the mucus metaplasia and the IL-13-independence of the other components of the VEGF phenotype.

### Example 5

### Effect on Antigen Sensitization, Th2 Inflammation and Dendritic Cells (DC)

### Evaluation of Antigen Sensitization

The effects of transgenic VEGF, transgenic L-11 or recombinant (r) VEGF₁₆₅ were evaluated. In the former WT and TG mice were randomized to normal or dox water. Two weeks later mice were exposed to aerosolized OVA (2%, grade V, Sigma, St. Louis, Mo.) twice a day for 10 days. In the later, lightly sensitized WT mice received intranasal rVEGF (10 μg/mouse) or vehicle control and followed 20 minutes later by the same dose of OVA for 10 consecutive days. After an additional week representative animals were sacrificed and serum IgG₁ and IgG₂ₐ were quantitated as described (Park et al., 2001). The remaining animals received intranasal OVA (25 μg/mouse) and inflammation was assessed 48 hours later.

Splenocyte Proliferation Assay

Spleens were harvested 2 days after the last OVA challenge and triturated between sterile frosted slides. After red blood cell lysis, the splenocytes were washed, suspended in RPMI with 5% fetal bovine serum (Gibco), 1% L-glutamine, and 1% penicillin/streptomycin and incubated in media alone or with 1, 5, 10, 50, and 100 μg OVA/well at 5×10⁵ cells/well in 96-well flat-bottom plates at 37° C. for 48 hours. Keyhole limpet hemocyanin (KLH) (Pierce, Rockford, Ill.) was used as an antigen control. After 18 hours of culture with 1 μCi ³H thymidine, cellular thymidine incorporation was assessed.

DC Analysis

Single cell suspensions from lungs from WT and TG mice on normal or dox water were prepared as described previously (Vermaelen et al., 2003). After red blood cell lysis, FACS analysis was performed with the following mAbs obtained from BD Biosciences Pharmingen (San Diego, Calif.): anti-I-Abb-biotin, anti-CD3-FITC, anti-CD4-PE (GK1.5), anti-CD8α-PE (53-6.7), anti-CD11b-APC-Cy7 (M1/70), anti-CD11c-FITC, anti-CD40-PE, anti-CD80-PE, anti-CD86-PE, anti-B220-PE. PE-labeled anti-B7h/ICOS-L mAb were obtained from eBioscience. PE-conjugated rat IgG₂ₐ and rat IgG_{2b} were used as isotype controls. SAV-PerCP was utilized as a second step reagent for biotinylated anti-1-Abb. When necessary, cells were preincubated with anti-CD16/CD32 mAb to block cell surface Fc receptors. Cells gated by forward- and side-scatter parameters were analyzed on a FACScalibur flow cytometer (Becton Dickinson, San Jose, Calif.) using CELLQuest software.

Results:

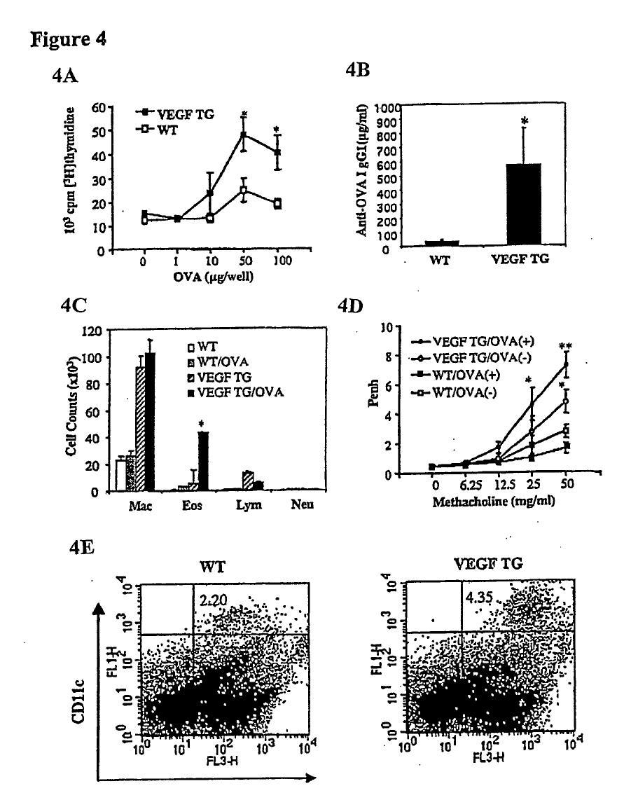

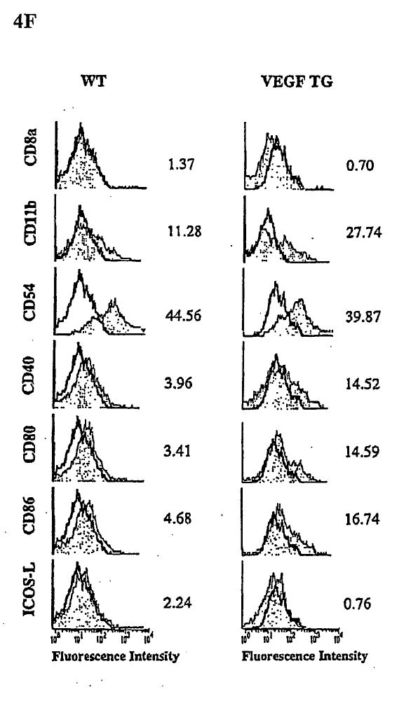

To determine if VEGF altered antigen sensitization via the respiratory tract, WT and TG mice were exposed to ovalbumin (OVA) by aerosol and systemic sensitization was assessed by quantitating OVA-induced splenocyte proliferation and the levels of OVA-specific IgG₁. OVA did not cause significant sensitization in WT mice on normal or dox water and TG mice on normal water. It did, however, engender a remarkable level of sensitivity in dox-treated TG animals (FIG. 4A-4B). In accord with this observation, TG mice manifest increased BAL and tissue eosinophilic inflammation (FIG. 4C) and enhanced AHR (FIG. 4D) after aerosol antigen sensitization and challenge. Similar results were obtained when rVEGF was administered with intranasal OVA to WT mice using a similar experimental protocol (data not shown). In both experimental systems, alterations in OVA-specific IgG₂ₐ and keyhole limpet hemocyanin (KLH)-induced splenocyte proliferation could not be appreciated (data not shown). In addition, enhanced respiratory antigen sensitization was not seen when CC10-IL-11 mice were challenged with aerosol OVA (data not shown). Thus, VEGF selectively increases Th2 antigen sensitization and inflammation in the lung.

The number and state of activation of parenchymal dendritic cells (DC) were similar in WT mice on normal or dox water and TG mice on normal water. In contrast, in TG animals, as little as 7 days of dox increased the number of CD11_{c}^{high}, MHC II^{high} DC in pulmonary tissues (FIG. 4E). These cells had increased levels of CD11b and decreased levels of CD8α (FIG. 4F). They were also activated because they expressed increased levels of MHC Class II, CD80/B7.1, CD86/B7.2 and CD40 and decreased levels of ICOSL/B.7h (FIG. 4F). Thus, VEGF increases the number and state of activation of DC2 cells in pulmonary tissues.

### Example 7

### Role(s) of VEGF in Th2 Inflammation

Role of VEGF in OVA-Stimulated Inflammation

The OVA model was undertaken in wild type mice as previously described (Wang et al., 2000). Sensitization and challenge were undertaken in the presence and absence of the VEGF receptor antagonist SU1498 (10 mg/kg/day) or VEGF Trap_{R1R2} (Regeneron, Inc., Tarrytown, N.Y.) (25 mg/kg) which were given via an intraperitoneal (IP) route starting the day before the first dose of OVA. The VEGF Trap_{R1R2} is a fusion protein comprised of the extracellular domains of VEGF_{R1} and VEGF_{R2} coupled to the Fc-portion of IgG that binds VEGF-A, but not VEGF-B or VEGF-C (Cursiefen et al., 2004). T-bet null mutant mice (Finotto et al., 2002) were obtained from the Jackson Laboratories (Bar Harbor, Me.). At 6 weeks of age they were randomized to SU1498 or control vehicle as described above. Two weeks later phenotypic characterization was undertaken as described above.

Th1 and Th2 Cell VEGF Production

As previously described (Cohn et al., 1997), CD4 T cells from OT-II mice that are transgenic for OVA-specific TCR were generated and polarized in vitro into Th1 or Th2 cells. They were then washed and incubated either (a) in the presence and absence of APC (syngeneic T-depleted splenocytes) and antigen (pOVA³²³⁻³³⁹, 15 μg/ml) for 24 hours or (b) with PMA (10 ng/ml, Sigma, St Louis, Mo.) and ionomycin (500 ng/ml, Sigma). Cell supernatant VEGF was quantitated by ELISA as described above.

Results:

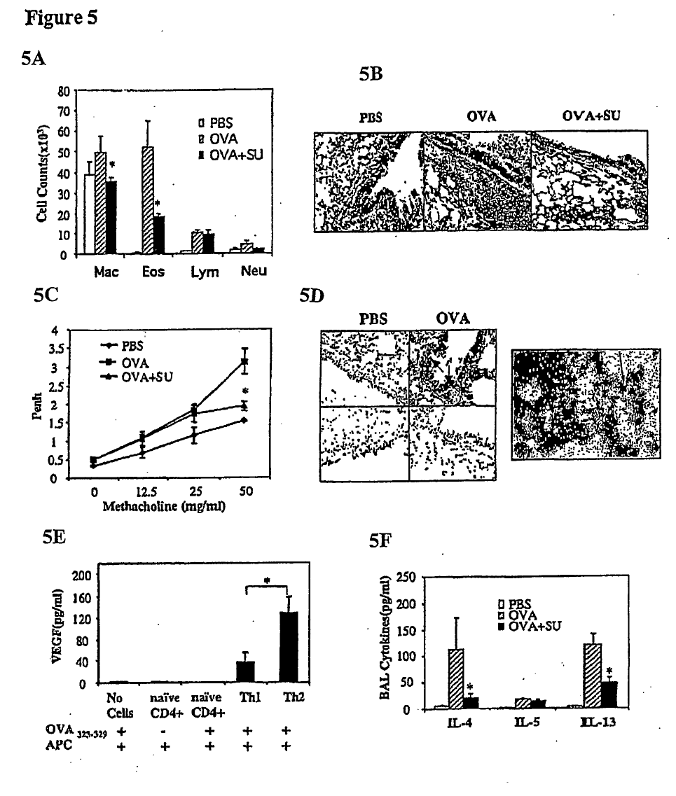

Studies were next undertaken to evaluate the role(s) of VEGF in antigen-induced Th2 inflammation. As previously reported (Wang et al., 2000), eosinophil-, lymphocyte- and macrophage-rich inflammation and AHR were seen in wild type mice sensitized and challenged with the aeroallergen ovalbumin (OVA) (FIG. 5A-5C and data not shown). This response was associated with increased levels of immunogenic VEGF in BAL fluids. Overall, BAL from control mice had 6.3+3 (range 5-15) pg/ml VEGF while OVA sensitized and challenged mice had 27.1+3 (range 20-035) pg/ml VEGF (p<0.01). This VEGF could be detected in airway epithelial cells including CC10 positive Clara cells and CD34+ T cells (FIG. 5D and data not shown). Studies using in vitro polarized murine T cells also demonstrated that Th2 cells are potent producers of VEGF when compared to Th1 cells or naïve CD4 cells when stimulated with antigen in the presence of antigen-presenting cells (APC) or with PMA and ionomycin in the absence of APC (FIG. 5E and data not shown). Importantly, SU1498 markedly decreased BAL and tissue inflammation and AHR (FIG. 5A-5C). Similar results were seen with the VEGF_{R1R2} TRAP (FIG. 9). SU1498 and the TRAP also markedly decreased antigen-stimulated IL-13 and IL-4 production (FIG. 5F and FIG. 9). This response was not specific for OVA because VEGF induction was readily appreciated (FIG. 5G) and similar decreases in inflammation, and AHR were seen in T-bet null (Finotto et al., 2002) mice treated in a similar fashion (FIG. 5 and FIG. 9). Thus, VEGF is produced by epithelial cells and Th2 cells and plays a critical role in the pathogenesis of Th2 inflammation, cytokine elaboration and AHR.

### Example 8

### Reversibility of the VEGF Phenotype

Assessment of Reversibility

VEGF TG (+) mice were randomized to dox or normal water for 2-4 weeks and then placed on normal water. VEGF-induced alterations were assessed at the end of the dox administration interval and after 2-4 weeks of normal water as described above.

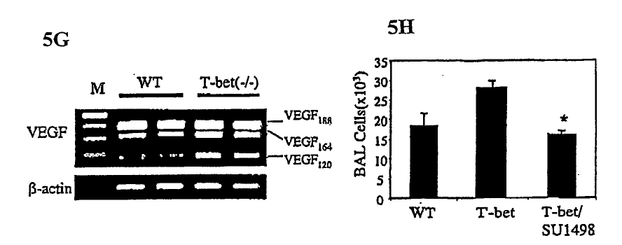

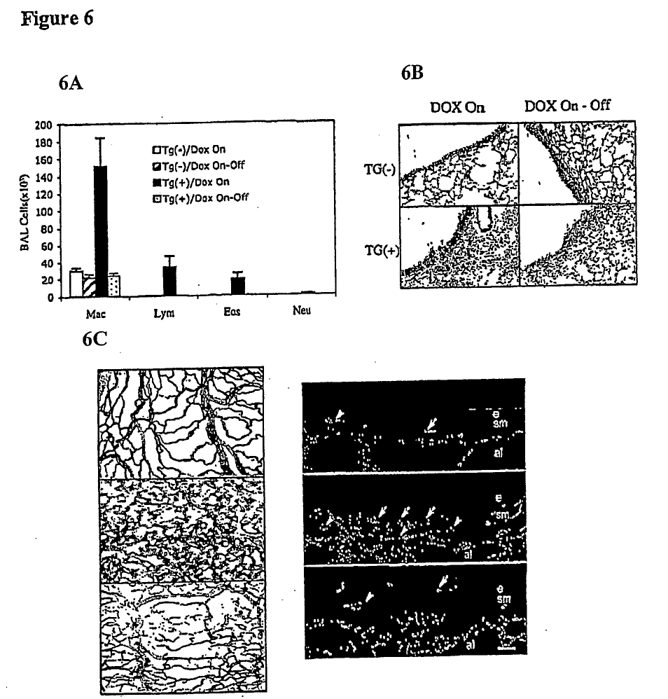

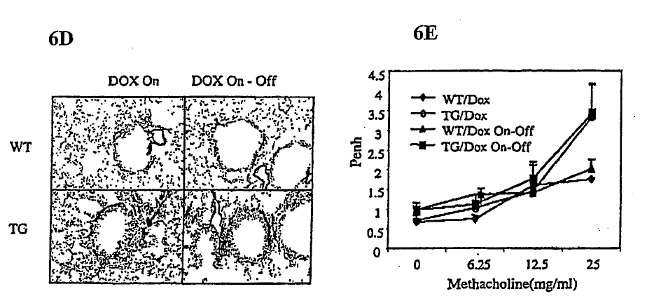

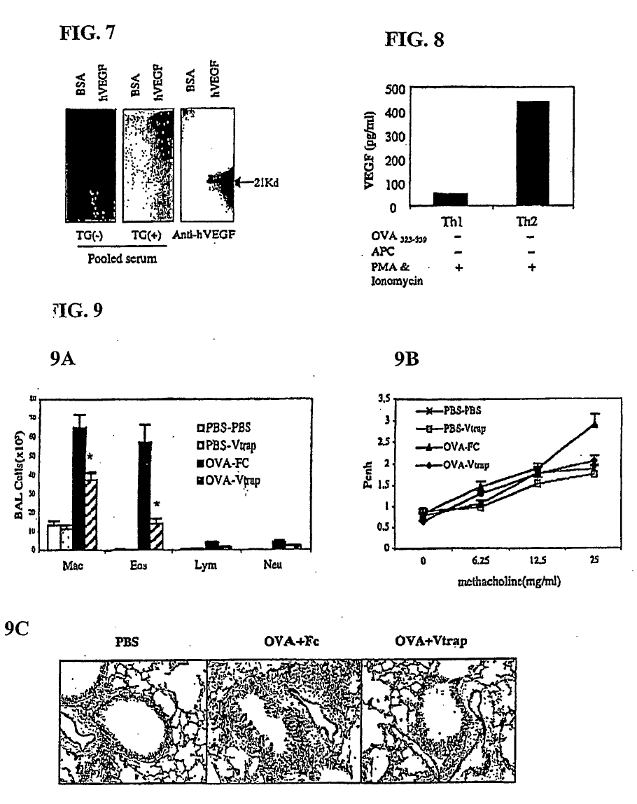

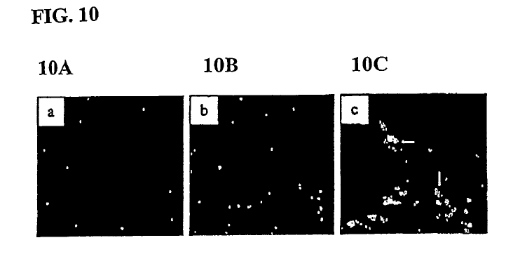

To define the reversibility of VEGF-induced phenotypic alterations, TG (+) mice and littermate controls received dox water for 2 weeks. The dox was then removed, transgenic VEGF production ceased and phenotypic characterization was undertaken. As noted above, transgenic VEGF was a potent stimulator of inflammation, vascular remodeling, mucus metaplasia, mucin gene expression, dendritic cell alterations, smooth muscle hyperplasia and AHR. Two weeks after the cessation of transgenic VEGF elaboration, BAL and tissue inflammation (FIG. 6A and data not shown), mucus metaplasia and enhanced mucin gene expression (FIG. 6B and data not shown) and angiogenesis (FIG. 6G) had returned to basal levels. During this interval, DC numbers also normalized and the level of DC activation returned to pre-dox levels (data not shown). In contrast, even 1 month after the cessation of transgene expression, smooth muscle hyperplasia and AHR were still readily apparent in lungs from TG (+) animals (FIGS. 6D-6E). These studies demonstrate that many VEGF-induced pulmonary alterations are readily reversible after the cessation of VEGF elaboration. They also demonstrate that VEGF-induced smooth muscle and physiologic abnormalities do not have the same propensity toward normalization.

Discussion

An increase in vessel size, number and surface area and the exaggerated expression of VEGF and VEGF receptors is well documented in the asthmatic airway (Vrugt et al., 2000; Salvato et al., 2001; Li et al., 1997; Lee et al., 2001; Orsida et al., 2001; Horshino et al., 2001; Kanazawa et al., 2002; Asai et al., 2002). Surprisingly, the mechanisms of asthmatic vascular remodeling and the vascular and non-vascular contributions of VEGF to asthma pathogenesis have not been defined. To address this issue lung-targeted VEGF₁₆₅ OE transgenic mice were generated and characterized and the role of VEGF in pulmonary Th2 inflammation was evaluated. These studies demonstrate, for the first time, that levels of VEGF that are in accord with those in human tissues and biologic fluids (Kanazawa et al., 2002; Asai et al., 2002; Ohta et al., 2002; Meyer et al., 2000; Nishigaki et al., 2003) induce an asthma-like phenotype characterized by inflammation, edema, angiogenesis, vascular remodeling, mucus metaplasia, subepithelial fibrosis, smooth muscle hyperplasia and AHR. They also demonstrate, for the first time, that VEGF increases antigen sensitization via the respiratory tract, augments antigen-induced Th2 inflammation, increases the accumulation and activation of pulmonary DC2 cells and plays a key role in antigen-induced Th2 inflammation and cytokine elaboration. Prior studies demonstrated that VEGF is produced during innate immune responses induced by RSV and endotoxin (Lee et al., 2000; Hahn et al., 2003). When combined these studies provide evidence that VEGF-plays a critical role in pulmonary Th2 inflammation and provide a potential explanation for many of the gaps in our present understanding of asthma pathogenesis.

First, these studies demonstrate that the VEGF produced during innate responses can generate asthma-like inflammation, airway and vascular remodeling and physiologic dysregulation. These findings demonstrate that, in contrast to the reports of Kaner et al. and Partovian and colleagues, VEGF is a potent mediator of vascular remodeling and angiogenesis in the normal lung. They also support the contention that VEGF is an important mediator of the vascular changes in asthmatic airways. Prior to this invention, one skilled in the art believed that asthmatic airway remodeling is caused by chronic Th2 inflammation. However, the present studies demonstrate that remodeling can also be caused by innate responses and highlight the importance of VEGF in the genesis of these alterations. This provides an explanation for the recent observation that airway remodeling can be seen in childhood asthma well before the ravages of chronic Th2 inflammation would be expected (Payne et al., 2003). Since elevated serum VEGF levels that correlate inversely with pulmonary function have been noted in patients with cystic fibrosis (McColley et al., 2000), VEGF most likely contributes to remodeling in this disorder as well.

Second, these studies demonstrate that VEGF augments Th2 antigen sensitization via the respiratory tract and simultaneously increases tissue permeability and the number and state of activation of pulmonary DC 2 cells. Thus, the ability of agents such as RSV and endotoxin to enhance antigen sensitization and Th2 inflammation (Schwarze et al., 2002; Eisenbarth et al., 2002) may be mediated by VEGF and that this induction may explain how RSV infection in early life contributes to the development of asthma (Schwarze et al., 2002; Sigurs et al., 2000). The exaggerated levels of airway VEGF in asthma may also contribute to the proclivity of asthmatics to become sensitized to respiratory antigens.

Based on the findings reported herein, VEGF can link innate and adaptive immunity by predisposing the lung (and possibly other organs) to antigen sensitization and, after antigen exposure, pathologic Th2 cytokine production and inflammation. These studies define a positive feedback loop with VEGF enhancing Th2 sensitization and inflammation and IL-13 subsequently enhancing VEGF elaboration. This interaction may contribute to the severity and or chronicity of VEGF or IL-13-mediated disorders.

Lastly, the studies demonstrate that, after sensitization, VEGF also plays a critical role in Th2 inflammation and cytokine elaboration. In the present invention we demonstrate that specific VEGF neutralization with a VEGF Trap abrogates antigen-induced inflammation and AHR. We also clarify the relevance of VEGF in classic aeroallergen-induced and genetic asthma models. Importantly we also define the mechanism of this inhibition by demonstrating that VEGF is induced in both models, that VEGF is produced by epithelial cells and T cells in the allergen-challenged lung, that VEGF is selectively elaborated by Th2 versus Th1 cells and that VEGF is required for antigen-induced Th2 cytokine elaboration. These effects of VEGF may relate to its ability to increase and activate DC2 cells.

The transgenic modeling system of the present invention is unique in its ability to assess the reversibility of transgene-induced pathologic responses. These studies demonstrate that VEGF-induced alterations differ in their degree of VEGF-dependence with inflammation, mucus metaplasia, angiogenesis and DC alterations reversing rapidly while smooth muscle hyperplasia and AHR did not reverse over an identical study interval. These findings provide evidence that therapies that inhibit VEGF will ameliorate inflammation, angiogenesis and mucus responses, even in patients with established disease.

In summary, these studies demonstrate that VEGF is a potent stimulator of inflammation, airway and vascular remodeling and physiologic dysregulation that augments antigen sensitization and Th2 inflammation and increases the number and activation of DC2 cells. They also demonstrate that these effects are mediated by IL-13-dependent and -independent pathways and highlight the impressive reversibility of some, but not all, of these VEGF-induced responses. Furthermore, they demonstrate that VEGF production is a critical event in Th2 inflammation and Th2 cytokine elaboration and that epithelial cells and Th2 cells are potent producers of VEGF in the antigen-challenged lung. Because VEGF can be induced during innate and adaptive immune responses these findings demonstrate how asthma-relevant responses can be induced by innate as well as adaptive inflammation. They also highlight mechanisms by which innate responses can predispose to antigen sensitization and Th2 inflammation. These findings provide a rationale for the use of VEGF regulators to prevent and or treat asthma and other Th2-dominated inflammatory disorders.

The foregoing detailed description has been given for clearness of understanding only and no unnecessary limitations should be understood therefrom as modifications will be obvious to those skilled in the art.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### REFERENCES

The disclosures of each and every patent, patent application, and publication cited herein including but limited to the references listed immediately below are hereby incorporated herein by reference in their entirety.
1. Wills-Karp, M. & Chiaramonte, M. Interleukin-13 in asthma. Curr Opin Pulm Med 9, 21-7. (2003).
2. Elias, J. A., Zhu, Z., Chupp, G. & Horner, R. J. Airway remodeling in asthma. J Clin Invest 104, 1001-6 (1999).
3. Elias, J. A. et al. New insights into the pathogenesis of asthma. J Clin Invest 111, 291-7 (2003).
4. Vrugt, B. et al. Bronchial angiogenesis in severe glucocorticoid-dependent asthma. Eur Respir J 15, 1014-21 (2000).
5. Salvato, G. Quantitative and morphological analysis of the vascular bed in bronchial biopsy specimens from asthmatic and non-asthmatic subjects. Thorax 56, 902-6 (2001).
6. Li, X. & Wilson, J. W. Increased vascularity of the bronchial mucosa in mild asthma. Am J Respir Crit Care Med 156, 229-33 (1997).
7. Lee, Y. C. & Lee, H. K. Vascular endothelial growth factor in patients with acute asthma. J. Allergy Clin. Immunol. 107, 1106-1108 (2001).
8. Orsida, B. E. et al. Effect of a long-acting beta2-agonist over three months on airway wall vascular remodeling in asthma. Am J Respir Crit Care Med 164, 117-21 (2001).
9. Hogg, J. C. Vascularity in asthmatic airways: Relation to inhaled steroid dose. Thorax 54, 283 (1999).
10. Hoshino, M., Nakamura, Y. & Hamid, Q. A. Gene expression of vascular endothelial growth factor and its receptors and angiogenesis in bronchial asthma. Allergy Clin. Immunol. 107, 1034-1038 (2001).
11. Hoshino, M., Takahashi, M. & Aoike, N. Expression of vascular endothelial growth factor, basic fibroblast growth factor, and angiogenin immunoreactivity in asthmatic airways and its relationship to angiogenesis. J Allergy Clin Immunol 107, 295-301 (2001).
12. Charan, N. B., Baile, E. M. & Pare, P. D. Bronchial vascular congestion and angiogenesis. Eur Respir J 10, 1173-80 (1997).
13. Thurston, G. et al. Angiopoietin-1 protects the adult vasculature against plasma leakage. Nat Med 6, 460-3 (2000).
14. Antony, A. B., Tepper, R. S. & Mohammed, K. A. Cockroach extract antigen increases bronchial airway epithelial permeability. J Allergy Clin Immunol 110, 589-95 (2002).
15. Senger, D. R. et al. Vascular permeability factor (VPF, VEGF) in tumor biology. Cancer Metastasis Rev 12, 303-24 (1993).
16. Gerber, H. P., Dixit, V. & Ferrara, N. Vascular endothelial growth factor induces expression of the antiapoptotic proteins Bcl-2 and A1 in vascular endothelial cells. J Biol Chem 273, 13313-6 (1998).
17. Clauss, M. Molecular biology of the VEGF and the VEGF receptor family. Semin Thromb Hemost 26, 561-9 (2000).
18. Kanazawa, H., Hirata, K. & Yoshikawa, J. Involvement of vascular endothelial growth factor in exercise induced bronchoconstriction in asthmatic patients. Thorax 57, 885-8 (2002).
19. Asai, K. et al. Imbalance between vascular endothelial growth factor and endostatin levels in induced sputum from asthmatic subjects. J Allergy Clin Immunol 110, 571-5 (2002).
20. Kaner, R. J. et al. Lung overexpression of the vascular endothelial growth factor gene induces pulmonary edema. Am J Respir Cell Mol Biol 22, 657-64 (2000).
21. Partovian, C. et al. Adenovirus-mediated lung vascular endothelial growth factor overexpression protects against hypoxic pulmonary hypertension in rats. Am J Respir Cell Mol Biol 23, 762-71 (2000).
22. Schwarze, J. & Gelfand, E. W. Respiratory viral infections as promoters of allergic sensitization and asthma in animal models. Eur. Respir. J. 19, 341-349 (2002).
23. Eisenbarth, S. C. et al. Lipopolysaccharide-enhanced, toll-like receptor 4-dependent T helper cell type 2 responses to inhaled antigen. J Exp Med 196, 1645-51 (2002).
24. Umetsu, D. T., McIntire, J. J., Akbari, O., Macaubas, C. & DeKruyff, R. H. Asthma: an epidemic of dysregulated immunity. Nat Immunol 3, 715-20 (2002).
25. Sigurs, N., Bjarnason, R., Sigurbergsson, F. & Kjellman, B. Respiratory syncytial virus bronchiolitis in infancy is an important risk factor for asthma and allergy at age 7. Am J Respir Crit Care Med 161, 1501-7 (2000).
26. Lee, C. G. et al. Respiratory syncytial virus stimulation of vascular endothelial cell growth Factor/Vascular permeability factor. Am J Respir Cell Mol Biol 23, 662-9 (2000).
27. Zeng, X., Wert, S. E., Federici, R., Peters, K. G. & Whitsett, J. A. VEGF enhances pulmonary vasculogenesis and disrupts lung morphogenesis in vivo. Dev Dyn 211, 215-27 (1998).
28. Zheng, T. et al. Inducible targeting of IL-13 to the adult lung causes matrix metalloproteinase- and cathepsin-dependent emphysema. J Clin Invest 106, 1081-93 (2000).
29. Ray, P. et al. Regulated overexpression of interleukin 11 in the lung. Use to dissociate development-dependent and -independent phenotypes. J Clin Invest 100, 2501-11 (1997).
30. Ohta, Y. et al. Vascular endothelial growth factor expression in airways of patients with lung cancer: a possible diagnostic tool of responsive angiogenic status on the host side. Chest 121, 1624-7 (2002).
31. Meyer, K. C., Cardoni, A. & Xiang, Z. Z. Vascular endothelial growth factor in bronchioalveolar lavage from normal subjects and patients with diffuse parenchymal lung disease. J Lab Clin Med 135, 332-8 (2000).
32. Nishigaki, Y. et al. Increased vascular endothelial growth factor in acute eosinophilic pneumonia. Eur Respir J 21, 774-8 (2003).
33. Wang, J. et al. IL-11 selectively inhibits aeroallergen-induced pulmonary eosinophilia and Th2 cytokine production. J Immunol 165, 2222-31 (2000).
34. Finotto, S. et al. Development of spontaneous airway changes consistent with human asthma in mice lacking T-bet. Science 295, 336-8 (2002).
35. Hahn, R. G. Endotoxin boosts the vascular endothelial growth factor (VEGF) in rabbits. J Endotoxin Res 9, 97-100 (2003).
36. Payne, D. N. et al. Early thickening of the reticular basement membrane in children with difficult asthma. Am J Respir Crit Care Med 167, 78-82 (2003).
37. McColley, S. A., Stellmach, V., Boas, S. R., Jain, M. & Crawford, S. E. Serum vascular endothelial growth factor is elevated in cystic fibrosis and decreases with treatment of acute pulmonary exacerbation. Am J Respir Crit Care Med 161, 1877-80 (2000).
38. Matsui, E. C. et al. Cockroach allergen exposure and sensitization in suburban middle-class children with asthma. J Allergy Clin Immunol 112, 87-92 (2003).
39. Corne, J. et al. IL-13 stimulates vascular endothelial cell growth factor and protects against hyperoxic acute lung injury. J Clin Invest 106, 783-91. (2000).
40. Lee, Y. C., Kwak, Y.-G. & Song, C. H. Contribution of vascular endothelial growth factor to airway hyperresponsiveness and inflammation in a murine model of toluene diisocyanate-induced asthma. J Immunol. 168, 3595-3600 (2002).
41. Vermaelen, K. Y. & Pauwels, R. A. Accelerated airway dendritic cell maturation, trafficking and elimination in a mouse model of asthma. Am J Respir Cell Mol Biol (2003).
42. Gabrilovich, D. I., Ishida, T., Nadaf, S., Ohm, J. E. & Carbone, D. P. Antibodies to vascular endothelial growth factor enhance the efficacy of cancer immunotherapy by improving endogenous dendritic cell function. Clin Cancer Res 5, 2963-70 (1999).
43. Matsuyama, W. et al. Purified protein derivative of tuberculin upregulates the expression of vascular endothelial growth factor in T lymphocytes in vitro. Immunology 106, 96-101 (2002).
44. Lee, C. G. et al. Transgenic overexpression of interleukin (IL)-10 in the lung causes mucus metaplasia, tissue inflammation, and airway remodeling via IL-13-dependent and -independent pathways. J Biol Chem 277, 35466-74 (2002).
45. Lee, C. G. et al. Interleukin-13 induces tissue fibrosis by selectively stimulating and activating TGF-β₁. J. Exp. Med. 194, 809-821 (2001).
46. Zhu, Z. et al. Pulmonary expression of interleukin-13 causes inflammation, mucus hypersecretion, subepithelial fibrosis, physiologic abnormalities and eotaxin production. J. Clin. Invest. 103, 779-788 (1999).
47. Park, Y. et al. The enhanced effect of a hexameric deoxyriboguanosine run conjugation to CpG oligodeoxynucleotides on protection against allergic asthma. J Allergy Clin Immunol 108, 570-6 (2001).
48. Thurston, G., Baluk, P., Hirata, A. & McDonald, D. M. Permeability related changes revealed at endothelial cell borders in inflamed vessels by lectin staining. Am J Physiol 271, H2547-H2562 (1996).
49. Baluk, P., Bolton, P., Hirata, A., Thurston, G. & McDonald, D. M. Endothelial gaps and adherent leukocytes in early and late phase plasma leakage in rat airways. Am. J. Pathol. 152, 1463-1476 (1998).
50. Cursiefen, C. et al. VEGF-A stimulates lymphangiogenesis and hemangiogenesis in inflammatory neovascularization via macrophage recruitment. J Clin Invest 113, 1040-50 (2004).
51. Cohn, L., Horner, R. J., Marinov, A., Rankin, J. & Bottomly, K. Induction of airway mucus production by T helper 2 (Th2) cells: a critical role for interleukin 4 in cell recruitment but not mucus production. J. Exp. Med. 186, 1737-1747 (1997).

What is claimed:

1. A method of treating or preventing a Th2 mediated inflammatory disease in a subject wherein said disease is associated with an increased level of VEGF, comprising administering an effective amount of a VEGF antagonist to said subject, thereby treating said inflammatory disease in said subject.

2. The method of claim 1, wherein said Th2 mediated inflammatory disease is asthma, chronic obstructive pulmonary disease, interstitial lung disease, chronic bronchitis, eosinophilic bronchitis, eosinophilic pneumonia, pneumonia, atopy, allergy, atopic dermatitis, allergic rhinitis, idiopathic pulmonary fibrosis and scleroderma.

3. The method of claim 2 wherein said Th2 mediated inflammatory disease is asthma not induced by exercise or toluene diisocyanate (TDI).

4. The method of claim 3, wherein said disease is characterized by pulmonary neovascularization.

5. The method of claim 3, wherein said disease is characterized by pulmonary angiogenesis.

6. The method of claim 3, wherein said disease is characterized by inflammation, parenchymal and vascular remodeling, edema, mucus metaplasia, myocyte hyperplasia, and airways hyperresponsiveness.

7. The method of claim 1, wherein said VEGF antagonist is selected from the group consisting of a chemical compound, an antibody, a VEGF trap, a ribozyme, a nucleic acid, a peptide, an antisense nucleic acid molecule, and an interfering RNA (RNAi) molecule.

8. The method of claim 7, wherein said chemical compound is selected from the group consisting of SU 1498 and SU 5614.

9. The method of claim 7, wherein said VEGF trap is a VEGF_{R1R2} Trap.

10. The method of claim 7, wherein said antisense nucleic acid molecule is an isolated nucleic acid complementary to an isolated nucleic acid encoding said VEGF, or a fragment thereof.

11. The method of claim 7, wherein said ribozyme is an isolated enzymatic nucleic acid, which specifically cleaves mRNA transcribed from a nucleic acid encoding said VEGF.

12. A method for treating a Th2 inflammatory disease in a subject wherein said disease is associated with an increased level of interleukin-13, said method comprising administering an effective amount of a VEGF antagonist to said subject, thereby treating said inflammatory disease in a subject.

13. The method of claim 12, wherein said disease is mucus metaplasia.

14. The method of claim 13, wherein said VEGF antagonist is selected from the group consisting of a chemical compound, an antibody, a VEGF trap, a ribozyme, a nucleic acid, a peptide, an antisense nucleic acid molecule and an interfering RNA molecule.

15. A method of identifying a compound useful for treating a Th2 mediated inflammatory disease in a mammal, said method comprising administering a compound to a mammal afflicted with the inflammatory disease and comparing the level of VEGF in said mammal with the level of said VEGF in said mammal prior to administration of said compound, wherein a lower level of said VEGF in said mammal after administration of said compound compared with said level of said VEGF in said mammal prior to administration of said compound is an indication that said compound is useful for treating an inflammatory disease in said mammal, thereby identifying a compound useful for treating an inflammatory disease.

16. The method of claim 15, wherein said level of VEGF is selected from the group consisting of the level of VEGF nucleic acid expression and the level of VEGF activity.

17. The method of claim 16, wherein said mammal is a mouse.

18. The method of claim 17, wherein said mouse is selected from the group consisting of a transgenic mouse inducibly expressing VEGF.

19. A method of identifying a compound useful for treating a Th2 mediated inflammatory disease, said method comprising contacting a cell or tissue with a compound and comparing the level or activity of VEGF in said cell or tissue with the level or activity of said VEGF in an otherwise identical cell or tissue not contacted with said compound, wherein a lower level or activity of said VEGF in said cell or tissue contacted with said compound compared with said level or activity of said VEGF in said cell or tissue not contacted with said compound is an indication that said compound is useful for treating a Th2 mediated inflammatory disease, thereby identifying a compound useful for treating a Th2 mediated inflammatory disease.
